(19) United States
(12) Patent Application Publication
(10) Pub. No.: US 2026/0193601 A1
(43) Pub. Date: Jul. 9, 2026

(54) COMPOSITION FOR CELL CULTURE

(71) Applicants: JW THERAPEUTICS R & D (SHANGHAI) CO., LTD., Shanghai (CN); JW THERAPEUTICS (SHANGHAI) CO., LTD., Shanghai (CN)

(72) Inventors: Wucheng WANG, Shanghai (CN); Yurun GAN, Shanghai (CN); Jinxiu LI, Shanghai (CN); Ting WANG, Shanghai (CN)

(21) Appl. No.: 19/133,745

(22) PCT Filed: Dec. 1, 2023
(86) PCT No.: PCT/CN2023/135949

§ 371 Date: May 29, 2025

(30) Foreign Application Priority Data
Dec. 2, 2022 (CN) 202211543352.2
Nov. 22, 2023 (CN) 202311573323.5

(51) Int. Cl. C12N 5/00

(52) U.S. Cl. CPC C12N 5/0031; C12N 2500/32; C12N 2500/99; C12N 2501/2302; C12N 2501/2307; C12N 2501/2315

(57) ABSTRACT

The present disclosure relates to a composition for cell culture, a method for preparing the composition, a method for culturing cells using the composition, and cells obtained thereby. Using the composition for cell culture disclosed herein to culture cells can achieve effects including large-scale cell expansion, maintaining high cell viability, an early memory phenotype, and a low exhaustion phenotype.

The present application claims priority to Chinese patent application 2022115433522 filed on Dec. 2, 2022 and Chinese patent application 2023115733235 filed on Nov. 22, 2023. The contents of the Chinese patent applications are incorporated herein by reference in their entireties.

### TECHNICAL FIELD

The present disclosure relates to a composition for cell culture, a method for preparing the composition, a method for culturing cells using the composition, and cells obtained thereby.

### BACKGROUND

A variety of cell therapy methods have been employed for treating diseases. The acquisition of cells that have not been or have been genetically engineered requires efficient cell culture processes, including media capable of achieving large-scale cell expansion while maintaining high cell viability, an early memory phenotype, and a low exhaustion phenotype. The present disclosure provides compositions and methods thereof fulfilling such need.

### SUMMARY

The present disclosure provides a composition capable of efficiently expanding cells. In one aspect, the composition comprises a first serum-free medium and a second serum-free medium at a volume ratio of 1:0.1 to 10; the composition exhibits a higher fold for expansion of immune cells than either the first serum-free medium or the second serum-free medium. In some embodiments, the cells are cultured for at least about 2, 3, 4, 5, 6, 7, 8, 9, 10, 11, 12, or 13 days. In some embodiments, the cells are cultured at 37.0±1.0° C. with 5.0±1.0% CO₂. In some embodiments, after culturing T cells with the composition for 9, 11, or 13 days, the fold expansion is increased by at least 50%, 60%, 70%, 80%, 90%, 100%, 150%, 200%, 250%, 300%, 350%, or 400% or more compared to a single serum-free medium. In some embodiments, the sum of the volumes of the first serum-free medium and the second serum-free medium accounts for 50% or more of the volume of the composition. In some embodiments, the sum of the volumes of the first serum-free medium and the second serum-free medium accounts for 65% or more of the volume of the composition. In some embodiments, the sum of the volumes of the first serum-free medium and the second serum-free medium accounts for 80% or more of the volume of the composition. In some embodiments, the composition consists of a first serum-free medium and a second serum-free medium. In some embodiments, the first serum-free medium is TexMACS; the second serum-free medium comprises one or more media selected from the group consisting of X-VIVO 10, X-VIVO 15, and X-VIVO 20. In some embodiments, the first serum-free medium is TexMACS; the second serum-free medium is X-VIVO 15. In some embodiments, the composition further comprises a third serum-free medium, wherein the third serum-free medium is AIM-V.

In one aspect, the present disclosure provides a composition comprising a medium substrate, wherein the medium substrate comprises a first serum-free medium and a second serum-free medium; the sum of the volumes of the first serum-free medium and the second serum-free medium accounts for 50% or more of the volume of the medium substrate; the volume ratio of the first serum-free medium to the second serum-free medium is 1:0.1 to 10; the first serum-free medium is TexMACS; the second serum-free medium comprises one or more media selected from the group consisting of X-VIVO 10, X-VIVO 15, and X-VIVO 20. In some embodiments, the sum of the volumes of the first serum-free medium and the second serum-free medium accounts for 65% or more of the volume of the medium substrate. In some embodiments, the medium substrate consists of a first serum-free medium and a second serum-free medium. In some embodiments, the first serum-free medium is TexMACS; the second serum-free medium is X-VIVO 15.

In some embodiments, the composition comprises a medium substrate, wherein the medium substrate comprises a first serum-free medium and a second serum-free medium; the sum of the volumes of the first serum-free medium and the second serum-free medium accounts for 80% or more of the volume of the medium substrate; the volume ratio of the first serum-free medium to the second serum-free medium is 1:0.1 to 10; the first serum-free medium is TexMACS; the second serum-free medium comprises one or more media selected from the group consisting of X-VIVO 10, X-VIVO 15, and X-VIVO 20.

In some embodiments, the composition comprises a medium substrate, wherein the medium substrate consists of a first serum-free medium and a second serum-free medium; the volume ratio of the first serum-free medium to the second serum-free medium is 1:0.1 to 10; the first serum-free medium is TexMACS; the second serum-free medium consists of one or more media selected from the group consisting of X-VIVO 10, X-VIVO 15, and X-VIVO 20. In some embodiments, the composition comprises a medium substrate, wherein the medium substrate consists of a first serum-free medium and a second serum-free medium; the volume ratio of the first serum-free medium to the second serum-free medium is 1:0.1 to 10; the first serum-free medium is TexMACS; the second serum-free medium is X-VIVO 15.

In some embodiments, the composition according to any one of the preceding embodiments, wherein the volume ratio of the first serum-free medium to the second serum-free medium is 1:0.1 to 2. In some embodiments, the volume ratio of the first serum-free medium to the second serum-free medium is 1:0.25 to 1. In some embodiments, the volume ratio of the first serum-free medium to the second serum-free medium is 1:0.1, 1:0.25, 1:0.5, or 1:1. In some embodiments, the volume ratio of the first serum-free medium to the second serum-free medium is about 1:0.5.

In some embodiments, the composition according to any one of the preceding embodiments comprises a medium substrate, wherein the medium substrate comprises TexMACS and X-VIVO 15; the volume of TexMACS and X-VIVO 15 accounts for 80% or more of the volume of the medium substrate; the volume ratio of TexMACS to X-VIVO 15 is 1:0.1 to 2. In some embodiments, the composition consists of TexMACS and X-VIVO 15 in a volume ratio of 1:0.25 to 1. In some embodiments, the composition consists of TexMACS and X-VIVO 15 in a volume ratio of 1:0.5.

In some embodiments, the composition according to any one of the preceding embodiments comprises a third serum-free medium AIM-V, wherein the ratio of the sum of the volumes of the first and second serum-free media to the volume of the third serum-free medium is not less than 2:1. In some embodiments, the ratio of the sum of the volumes of the first and second serum-free media to the volume of the third serum-free medium is not less than 4:1. In some embodiments, the ratio of the sum of the volumes of the first and second serum-free media to the volume of the third serum-free medium is not less than 10:1.

In some embodiments, the composition according to any one of the preceding embodiments, wherein the medium substrate comprises a third serum-free medium AIM-V; the ratio of the sum of the volumes of the first and second serum-free media to the volume of the third serum-free medium is not less than 2:1. In some embodiments, the ratio of the sum of the volumes of the first and second serum-free media to the volume of the third serum-free medium is not less than 4:1. In some embodiments, the ratio of the sum of the volumes of the first and second serum-free media to the volume of the third serum-free medium is not less than 10:1.

In some embodiments, the composition according to any one of the preceding embodiments, wherein the composition comprises a supplement added to the medium substrate; the supplement comprises one or more components selected from the group consisting of inorganic salts, sugars, vitamins, albumin, lipids, amino acids, cytokines, and antioxidants. In some embodiments, the supplement comprises one or more components selected from the group consisting of amino acids, cytokines, and antioxidants. In some embodiments, the supplement is or comprises amino acids and cytokines. In some embodiments, the supplement is or comprises amino acids, cytokines, and antioxidants.

In some embodiments, the composition according to any one of the preceding embodiments, wherein the cytokine comprises one or more components selected from the group consisting of IL-1, IL-2, IL-3, IL-4, IL-6, IL-7, IL-9, IL-10, IL-12, IL-13, IL-15, IL-17, IL-18, IL-21, IL-22, interferon, and tumor necrosis factor. In some embodiments, the cytokine comprises one or more components selected from the group consisting of IL-2, IL-7, and IL-15. In some embodiments, the cytokine is or comprises IL-2. In some embodiments, the cytokine is or comprises IL-2 and IL-7. In some embodiments, the cytokine is or comprises IL-7 and IL-15. In some embodiments, the cytokine is or comprises IL-2, IL-7, and IL-15.

In some embodiments, the composition according to any one of the preceding embodiments, wherein the concentration of the IL-2 supplementarily added in the composition is 10 to 1000 IU/mL. In some embodiments, the concentration of the IL-2 supplementarily added in the composition is 60 to 200 IU/mL. In some embodiments, the concentration of the IL-2 supplementarily added in the composition is about 100 IU/mL.

In some embodiments, the composition according to any one of the preceding embodiments, wherein the concentration of the IL-2 supplementarily added in the composition is 10 to 1000 IU/mL, the concentration of the IL-7 is 100 to 5000 IU/mL, and the concentration of the IL-15 is 0 to 500 IU/mL. In some embodiments, the concentration of the IL-2 supplementarily added in the composition is 60 to 200 IU/mL, the concentration of the IL-7 is 500 to 1000 IU/mL, and the concentration of the IL-15 is 25 to 100 IU/mL. In some embodiments, the concentration of the IL-2 supplementarily added in the composition is about 100 IU/mL, the concentration of the IL-7 is about 700 IU/mL, and the concentration of the IL-15 is about 46 IU/mL.

In some embodiments, the composition according to any one of the preceding embodiments, wherein the amino acid is L-alanyl-L-glutamine. In some embodiments, the concentration of the L-alanyl-L-glutamine supplementarily added in the composition is 1 mM to 5 mM. In some embodiments, the concentration of the L-alanyl-L-glutamine supplementarily added in the composition is about 2 mM.

In some embodiments, the composition according to any one of the preceding embodiments, wherein the antioxidant is N-acetyl-L-cysteine. In some embodiments, the concentration of the N-acetyl-L-cysteine supplementarily added in the composition is 0.5 mg/mL to 2 mg/mL. In some embodiments, the concentration of the N-acetyl-L-cysteine supplementarily added in the composition is 0.5 mg/mL to 1 mg/mL. In some embodiments, the concentration of the N-acetyl-L-cysteine supplementarily added in the composition is about 0.8 mg/mL.

In some embodiments, the composition according to any one of the preceding embodiments comprises a medium substrate, wherein the medium substrate comprises a first serum-free medium, a second serum-free medium, and optionally a third serum-free medium AIM-V; the sum of the volumes of the first serum-free medium and the second serum-free medium accounts for 80% or more of the volume of the medium substrate; the volume ratio of the first serum-free medium to the second serum-free medium is 1:0.25 to 1. In some embodiments, the medium substrate consists of a first serum-free medium and a second serum-free medium, wherein the volume ratio of the first serum-free medium to the second serum-free medium is 1:0.25 to 1. In some embodiments, the medium substrate consists of a first serum-free medium, a second serum-free medium, and a third serum-free medium, wherein the volume ratio of the first serum-free medium to the second serum-free medium is 1:0.25 to 1; the ratio of the sum of the volumes of the first and second serum-free media to the volume of the third serum-free medium is not less than 10:1. In some embodiments, the medium substrate consists of a first serum-free medium and a second serum-free medium, wherein the volume ratio of the first serum-free medium to the second serum-free medium is about 1:0.5. In some embodiments, the medium substrate consists of a first serum-free medium, a second serum-free medium, and a third serum-free medium, wherein the volume ratio of the first serum-free medium, the second serum-free medium, and the third serum-free medium is about 5:4:1.

In some embodiments, the composition according to any one of the preceding embodiments comprises a supplement added to the medium substrate, wherein the supplement comprises IL-2, IL-7, IL-15, L-alanyl-L-glutamine, and optionally N-acetyl-L-cysteine. In some embodiments, the concentration of the IL-2 supplementarily added in the composition is 50 to 200 IU/mL, the concentration of the IL-7 is 500 to 1000 IU/mL, the concentration of the IL-15 is 25 to 100 IU/mL, the concentration of the L-alanyl-L-glutamine is 1 mM to 5 mM, and the concentration of the N-acetyl-L-cysteine is 0.5 mg/mL to 2 mg/mL. In some embodiments, the concentration of the IL-2 supplementarily added in the composition is about 100 IU/mL, the concentration of the IL-7 is about 700 IU/mL, the concentration of the IL-15 is about 46 IU/mL, the concentration of the L-alanyl-L-glutamine is about 2 mM, and the concentration of the N-acetyl-L-cysteine is about 0.8 mg/mL.

In some embodiments, the composition according to any one of the preceding embodiments comprises a medium substrate and a supplement added to the medium substrate, wherein the medium substrate consists of TexMACS and X-VIVO 15 in a volume ratio of 1:0.25 to 1; the supplement comprises IL-2, IL-7, IL-15, and L-alanyl-L-glutamine, wherein the concentration of the IL-2 supplementarily added in the composition is 50 to 200 IU/mL, the concentration of the IL-7 is 500 to 1000 IU/mL, the concentration of the IL-15 is 25 to 100 IU/mL, and the concentration of the L-alanyl-L-glutamine is 1 mM to 5 mM.

In some embodiments, the composition according to any one of the preceding embodiments comprises a medium substrate and a supplement added to the medium substrate, wherein the medium substrate consists of TexMACS and X-VIVO 15 in a volume ratio of 1:0.25 to 1; the supplement comprises IL-2, IL-7, IL-15, L-alanyl-L-glutamine, and N-acetyl-L-cysteine, wherein the concentration of the IL-2 supplementarily added in the composition is 50 to 200 IU/mL, the concentration of the IL-7 is 500 to 1000 IU/mL, the concentration of the IL-15 is 25 to 100 IU/mL, the concentration of the L-alanyl-L-glutamine is 1 mM to 5 mM, and the concentration of the N-acetyl-L-cysteine is 0.5 mg/mL to 2 mg/mL.

In some embodiments, the composition according to any one of the preceding embodiments consists of a medium substrate, and IL-2, IL-7, IL-15, and L-alanyl-L-glutamine added to the medium substrate, wherein the medium substrate consists of TexMACS and X-VIVO 15 in a volume ratio of 1:0.5; the concentration of the IL-2 supplementarily added in the composition is about 100 IU/mL, the concentration of the IL-7 is about 700 IU/mL, the concentration of the IL-15 is about 46 IU/mL, and the concentration of the L-alanyl-L-glutamine is about 2 mM.

In some embodiments, the composition according to any one of the preceding embodiments consists of a medium substrate, and IL-2, IL-7, IL-15, L-alanyl-L-glutamine, and N-acetyl-L-cysteine added to the medium substrate, wherein the medium substrate consists of TexMACS and X-VIVO 15 in a volume ratio of 1:0.5; the concentration of the IL-2 supplementarily added in the composition is about 100 IU/mL, the concentration of the IL-7 is about 700 IU/mL, the concentration of the IL-15 is about 46 IU/mL, the concentration of the L-alanyl-L-glutamine is about 2 mM, and the concentration of the N-acetyl-L-cysteine is about 0.8 mg/mL.

In one aspect, the present disclosure provides a multicomponent composition comprising two or three selected from the group consisting of a first medium, a second medium, and a third medium. In some embodiments, the multicomponent composition comprises a first medium and a second medium. In some embodiments, the multicomponent composition comprises a first medium and a third medium. In some embodiments, the multicomponent composition comprises a second medium and a third medium. In some embodiments, the multicomponent composition comprises a first medium, a second medium, and a third medium. In some embodiments, the first medium comprises or consists of a medium substrate, the second medium comprises or consists of a medium substrate, cytokines, amino acids, and antioxidants, and the third medium comprises or consists of a medium substrate, cytokines, and amino acids.

In some embodiments, the multicomponent composition according to any one of the preceding embodiments, wherein the medium substrates of the first medium, the second medium, and the third medium each comprise a first serum-free medium and a second serum-free medium. In some embodiments, the first serum-free medium is TexMACS, and the second serum-free medium consists of one or more media selected from the group consisting of X-VIVO 10, X-VIVO 15, and X-VIVO 20. In some embodiments, the volume of the first serum-free medium and the second serum-free medium accounts for 80% or more of the volume of the medium substrate, and the volume ratio of the first serum-free medium to the second serum-free medium is 1:0.1 to 2. In some embodiments, the volume ratio of the first serum-free medium to the second serum-free medium is 1:0.25 to 1. In some embodiments, the volume ratio of the first serum-free medium to the second serum-free medium is 1:0.1, 1:0.25, 1:0.5, or 1:1.

In some embodiments, the first medium consists of TexMACS and X-VIVO 15 in a volume ratio of 1:0.25 to 1. In some embodiments, the volume ratio of TexMACS to X-VIVO 15 is 1:0.5.

In some embodiments, the multicomponent composition according to any one of the preceding embodiments, wherein the second medium comprises a medium substrate and a supplement added to the medium substrate. In some embodiments, the medium substrate of the second medium consists of TexMACS and X-VIVO 15 in a volume ratio of 1:0.25 to 1. In some embodiments, the volume ratio of TexMACS to X-VIVO 15 is 1:0.5. In some embodiments, the supplement comprises IL-2, IL-7, IL-15, L-alanyl-L-glutamine, and N-acetyl-L-cysteine, wherein the concentration of the IL-2 supplementarily added in the composition is 50 to 200 IU/mL, the concentration of the IL-7 is 500 to 1000 IU/mL, the concentration of the IL-15 is 25 to 100 IU/mL, the concentration of the L-alanyl-L-glutamine is 1 mM to 5 mM, and the concentration of the N-acetyl-L-cysteine is 0.5 mg/mL to 2 mg/mL. In some embodiments, the second medium consists of a medium substrate, and IL-2, IL-7, IL-15, L-alanyl-L-glutamine, and N-acetyl-L-cysteine added to the medium substrate, wherein the medium substrate consists of TexMACS and X-VIVO 15 in a volume ratio of 1:0.5; the concentration of the IL-2 supplementarily added in the composition is about 100 IU/mL, the concentration of the IL-7 is about 700 IU/mL, the concentration of the IL-15 is about 46 IU/mL, the concentration of the L-alanyl-L-glutamine is about 2 mM, and the concentration of the N-acetyl-L-cysteine is about 0.8 mg/mL.

In some embodiments, the multicomponent composition according to any one of the preceding embodiments, wherein the third medium comprises a medium substrate and a supplement added to the medium substrate. In some embodiments, the medium substrate of the third medium consists of TexMACS and X-VIVO 15 in a volume ratio of 1:0.25 to 1. In some embodiments, the volume ratio of TexMACS to X-VIVO 15 is 1:0.5. In some embodiments, the supplement comprises IL-2, IL-7, IL-15, and L-alanyl-L-glutamine, wherein the concentration of the IL-2 supplementarily added in the composition is 50 to 200 IU/mL, the concentration of the IL-7 is 500 to 1000 IU/mL, the concentration of the IL-15 is 25 to 100 IU/mL, and the concentration of the L-alanyl-L-glutamine is 1 mM to 5 mM. In some embodiments, the third medium consists of a medium substrate, and IL-2, IL-7, IL-15, and L-alanyl-L-glutamine added to the medium substrate, wherein the medium substrate consists of TexMACS and X-VIVO 15 in a volume ratio of 1:0.5; the concentration of the IL-2 supplementarily added in the composition is about 100 IU/mL, the concentration of the IL-7 is about 700 IU/mL, the concentration of the IL-15 is about 46 IU/mL, and the concentration of the L-alanyl-L-glutamine is about 2 mM.

In one aspect, the present disclosure provides a method for preparing a medium, comprising mixing the medium substrate according to any one of the preceding embodiments with a supplement.

In one aspect, the present disclosure provides a method for culturing cells, comprising incubating cells in the composition or multicomponent composition according to any one of the preceding embodiments.

In one aspect, the present disclosure provides a method for culturing cells, comprising a step of T cell expansion, wherein the step comprises expanding T cells in the third medium according to any one of the preceding embodiments. In some embodiments, the method for culturing cells comprises a step of T cell activation prior to T cell expansion, wherein the step comprises activating T cells in the second medium according to any one of the preceding embodiments. In some embodiments, the method for culturing cells comprises a step of T cell washing prior to T cell activation, wherein the step comprises washing T cells in the first medium according to any one of the preceding embodiments.

In one aspect, the present disclosure provides a method for culturing cells, comprising: 1) a step of cell isolation and washing, comprising washing T cells in the first medium according to any one of the preceding embodiments or DPBS; 2) a step of T cell activation, comprising activating T cells in the second medium according to any one of the preceding embodiments; and 3) a step of T cell expansion, comprising expanding T cells in the third medium according to any one of the preceding embodiments. In some embodiments, the step 2) comprises incubating the cells with magnetic beads conjugated to anti-CD3 antibody and anti-CD28 antibody in the second medium. In some embodiments, the method comprises a step of genetically engineering the T cells between the step 2) and the step 3).

In one aspect, the present disclosure provides a cell produced by the method for culturing cells according to any one of the preceding embodiments. In some embodiments, the cell is a mammalian cell or a human cell. In some embodiments, the cell is an immune cell, such as an innate or adaptive immune cell, e.g., a myeloid or lymphoid cell (including lymphocytes, e.g., T cells and/or NK cells). In some embodiments, the cell is a primary cell. In some embodiments, the cells are allogeneic and/or autologous. In some embodiments, the cells comprise one or more subsets of T cells or other cell types, such as the entire T cell population, CD4⁺ cells, CD8⁺ cells, and subpopulations thereof.

### BRIEF DESCRIPTION OF THE DRAWINGS

FIG. 1A shows the effect of cytokine concentration on cumulative fold expansion; FIG. 1B shows the effect of cytokine concentration on cell viability; FIG. 1C shows the effect of cytokine concentration on the proportion of T_{SCM}; FIG. 1D shows the effect of cytokine concentration on the proportion of PD-1 expression.

FIG. 2 shows the effect of the proportion of serum-free medium on T cell expansion.

### DETAILED DESCRIPTION OF THE PREFERRED EMBODIMENT

After reading this specification, it will be apparent to those skilled in the art how to implement the present disclosure in various alternative examples and alternative applications. However, the examples herein are presented by way of illustration only and not by way of limitation. As such, the detailed description should not be construed as limiting the scope or breadth of the present disclosure as set forth below.

### I. TERMINOLOGY

“A” or “an” may refer to one or more. “About”, “substantially”, and “approximately” refer to a range of increase or decrease of up to 5% relative to the corresponding numerical value. “Comprise”, “include”, and “contain” shall be construed as meaning that, in addition to the elements specified, any other feasible elements may be included, provided that the addition of such elements does not render the technical solution containing the elements unimplementable. “Consist of” refers to having only the elements described by “ . . . ”. “Comprise” covers the case of “consist of”.

The term “medium substrate” refers to a nutrient source used for growing or maintaining cells. The nutrient source contains essential components required for cell growth and/or survival and may be used separately for cell culture.

The term “serum-free medium” refers to a medium that is free or substantially free of serum. As used herein, “substantially free of serum” means containing less than about 1% by weight of serum, containing only trace amounts of serum, or containing undetectable amounts of serum.

The term “supplement” refers to a substance added in addition to the medium substrate for maintaining, activating, and/or expanding cells. The supplement herein may comprise one or more substances, the species of which may or may not have been included in the medium substrate. The concentration of the supplement herein refers to the concentration in addition to that of the corresponding substance in the medium substrate, e.g., if the total concentration of L-alanyl-L-glutamine in the composition is 5 mM and the original concentration of L-alanyl-L-glutamine in the medium substrate is 3 mM, then the concentration of L-alanyl-L-glutamine supplementarily added is 2 mM; if the total concentration of L-alanyl-L-glutamine in the composition is 2 mM and the medium substrate contains no L-alanyl-L-glutamine, then the concentration of L-alanyl-L-glutamine supplementarily added is 2 mM. The concentration of the supplement herein is calculated based on the original volume of the medium substrate.

The term “cytokine” refers to a biomolecule that affects cells of the immune system, including natural cytokines as well as fragments and functional variants thereof that have at least 10%, 30%, 50%, or 80% of the activity (e.g., immunomodulatory activity of naturally occurring cytokines) compared to the natural cytokines.

The term “amino acid” refers to all naturally occurring α-amino acids as well as synthetic oligopeptides, analogs, and derivatives thereof. An analog refers to the substitution of atoms in an amino acid for different atoms that typically exhibit similar properties. A derivative refers to a molecule in which an amino acid is attached to other structures, e.g., the product obtained by propionylation of the amino acid. The amino acid herein includes L-alanyl-L-glutamine.

The term “optionally” refers to presence or absence. For example, optionally N-acetyl-L-cysteine refers to the presence or absence of N-acetyl-L-cysteine in the supplement.

The term “culture” refers to maintaining cells in vitro under conditions conducive to growth and/or differentiation and/or sustained viability. “Culture” may be used interchangeably with “cell culture”.

The term “expansion” refers to the growth of cells in culture to increase the number of cells from an initial number of cells to a larger number of cells after culture.

The term “multicomponent composition” refers to a composition comprising two or more independent compositions, such as media.

The terms “immune cells” and “cells of the immune system” refer to cells involved in immune responses aimed at protecting an organism from foreign substances, viruses, and cells. Immune cells may be derived from various organs and tissues, such as the thymus, spleen, lymph nodes, and lymphoid tissue clusters (e.g., in the gastrointestinal tract and bone marrow). Such cells include T cells, B cells, natural killer cells, macrophages, neutrophils, tumor-infiltrating lymphocytes, dendritic cells, mast cells, eosinophils, and basophils, as well as progenitor cells that develop into these cells.

### II. MEDIUM

The present disclosure provides a composition comprising at least two serum-free culture media. Surprisingly, the composition of the present disclosure, when used for culturing immune cells (e.g., T cells), exhibits a significantly increased fold expansion of cells, more sustainable cell viability, a lower proportion of PD-1 expression, a higher proportion of CD25⁺CD69⁺ T cells, and/or a higher proportion of T_{SCM} compared to a single serum-free medium. For example, after culturing T cells with the composition for 7, 9, 11, or 13 days, the fold expansion is increased by 50%, 60%, 70%, 80%, 90%, 100%, 150%, 200%, 250%, 300%, 350%, or 400% or more compared to a single serum-free medium.

II-a. Medium Substrate

In some embodiments, the composition as described above comprises a medium substrate. In some embodiments, the medium substrate comprises a first serum-free medium and a second serum-free medium that are distinct from each other. In some embodiments, the serum-free medium is well known to those skilled in the art and commercially available, such as RPMI 1640, AIM-V, DMEM, MEM, a-MEM, F-12, TexMACS™, X-VIVO™10, X-VIVO™15, X-VIVO™20, or OpTmizer™. In some embodiments, the first serum-free medium is TexMACS™, and the second serum-free medium comprises one or more media selected from the group consisting of RPMI 1640, AIM-V, DMEM, MEM, a-MEM, F-12, X-VIVO™10, X-VIVO™15, X-VIVO™20, and OpTmizer™. In some embodiments, the first serum-free medium is TexMACS™, and the second serum-free medium comprises one or more media selected from the group consisting of X-VIVO™10, X-VIVO™15, and X-VIVO™20. In specific embodiments, the first serum-free medium is TexMACS™, and the second serum-free medium is AIM-V. In specific embodiments, the first serum-free medium is TexMACS™, and the second serum-free medium is F-12. In specific embodiments, the first serum-free medium is TexMACS™, and the second serum-free medium is X-RPMI 1640. In specific embodiments, the first serum-free medium is TexMACS™, and the second serum-free medium is DMEM. In specific embodiments, the first serum-free medium is TexMACS™, and the second serum-free medium is X-VIVO™15. In some embodiments, the first serum-free medium and the second serum-free medium in the composition are to be formulated or have been formulated into a medium.

In some embodiments, the volume of the first serum-free medium and the second serum-free medium as described above accounts for 50% or more, 55% or more, 60% or more, 65% or more, 70% or more, 75% or more, 80% or more, 85% or more, 90% or more, 95% or more, 96% or more, 97% or more, 98% or more, or 99% or more of the volume of the medium substrate. The volume of the first serum-free medium and the second serum-free medium accounts for 50%, 55%, 60%, 65%, 70%, 75%, 80%, 85%, 90%, 95%, 96%, 97%, 98%, 99%, or 100% of the volume of the medium substrate. In specific embodiments, the volume of the first serum-free medium and the second serum-free medium accounts for 100% of the volume of the medium substrate, i.e., the medium substrate consists of the first serum-free medium and the second serum-free medium. Those skilled in the art will appreciate that the volume after mixing different media and other solutions may slightly differ from the sum of the volumes before mixing. When there is a difference, the volume of the medium substrate herein refers to the sum of the volumes of the different media and other solutions before mixing.

In some embodiments, the volume ratio (v:v) of the first serum-free medium to the second serum-free medium as described above is 1:0.1 to 10, 1:0.2 to 5, 1:0.2 to 4, 1:0.2 to 3, 1:0.2 to 2, 1:0.2 to 1, 1:0.25 to 2, 1:0.25 to 1, 1:0.3 to 3, 1:0.3 to 2, 1:0.3 to 1, 1:0.4 to 2, 1:0.4 to 1, 1:0.5 to 2, or 1:0.5 to 1. In some embodiments, the volume ratio of the first serum-free medium to the second serum-free medium is or is about 1:1 or 1:0.5. In some embodiments, the volume of the first serum-free medium and the second serum-free medium accounts for 100% of the volume of the medium substrate, and the volume ratio of the first serum-free medium to the second serum-free medium is 1:0.1 to 10, 1:0.2 to 5, 1:0.2 to 4, 1:0.2 to 3, 1:0.2 to 2, 1:0.2 to 1, 1:0.25 to 2, 1:0.25 to 1, 1:0.3 to 3, 1:0.3 to 2, 1:0.3 to 1, 1:0.4 to 2, 1:0.4 to 1, 1:0.5 to 2, or 1:0.5 to 1. In some embodiments, the volume of the first serum-free medium and the second serum-free medium accounts for 100% of the volume of the medium substrate, and the volume ratio of the first serum-free medium to the second serum-free medium is or is about 1:1, 1:0.5, or 1:0.25. Herein, the volume ratio of the first serum-free medium to the second serum-free medium refers to the volume ratio before mixing.

In some embodiments, the medium substrate comprises a first serum-free medium, a second serum-free medium, and a third serum-free medium that are distinct from each other, wherein the first serum-free medium is TexMACS™, the second serum-free medium consists of one or more media selected from the group consisting of X-VIVO™10, X-VIVO™15, and X-VIVO™20, and the third serum-free medium is AIM-V. In some embodiments, the volume of the first, second, and third serum-free media as described above accounts for 50% or more, 55% or more, 60% or more, 65% or more, 70% or more, 75% or more, 80% or more, 85% or more, 90% or more, 95% or more, 96% or more, 97% or more, 98% or more, or 99% or more of the volume of the medium substrate. In some embodiments, the volume of the first, second, and third serum-free media accounts for 100% of the volume of the medium substrate. In some embodiments, the ratio of the sum of the volumes of the first and second serum-free media to the volume of the third serum-free medium is not less than 2:1, 3:1, 4:1, 5:1, 6:1, 7:1, 8:1, 9:1, or 10:1. In specific embodiments, the volume ratio of the first serum-free medium, the second serum-free medium, and the third serum-free medium is about 5:4:1.

II-b. Supplement

In some embodiments, the composition as described above comprises a supplement added to the medium substrate, wherein the supplement comprises one or more components selected from the group consisting of inorganic salts, sugars, vitamins, albumin, lipids, amino acids, stimulants (e.g., cytokines), and antioxidants. In some embodiments, the supplement comprises one or more components selected from the group consisting of amino acids, cytokines, and antioxidants. In some embodiments, the supplement is or comprises amino acids, cytokines, and antioxidants.

In some embodiments, the supplement as described above comprises a cytokine. In some embodiments, the cytokine is a recombinant cytokine. In specific embodiments, the cytokine is a recombinant human cytokine. In some embodiments, the cytokine is capable of binding to a receptor expressed by an immune cell (e.g., a T cell). In some embodiments, the cytokine comprises interleukin-2 (IL-2), interleukin-4 (IL-4), interleukin-7 (IL-7), interleukin-9 (IL-9), interleukin-12 (IL-12), interleukin-15 (IL-15), granulocyte colony-stimulating factor (G-CSF), and granulocyte-macrophage colony-stimulating factor (GM-CSF). In some embodiments, the cytokine comprises interferon and tumor necrosis factor. In some embodiments, the cytokine comprises one or more components selected from the group consisting of IL-2, IL-7, and IL-15. In some embodiments, the cytokine is or comprises IL-2. In specific embodiments, the cytokine is or comprises IL-2, IL-7, and IL-15. These cytokines are used as stimulants in the medium to activate the cells. When the cells have been activated, a medium without cytokines may be used.

In some embodiments, the cytokine as described above comprises IL-2. In some embodiments, the concentration of the IL-2 (e.g., human recombinant IL-2) is 10 to 1000 IU/mL, 50 to 500 IU/mL, 50 to 200 IU/mL, or 50 to 150 IU/mL. In specific embodiments, the concentration of the IL-2 is or is about 50 IU/mL, 60 IU/mL, 70 IU/mL, 80 IU/mL, 90 IU/mL, 100 IU/mL, 110 IU/mL, 120 IU/mL, 130 IU/mL, 140 IU/mL, or 150 IU/mL. In some embodiments, the concentration of the IL-2 is or is about 100 IU/mL.

In some embodiments, the cytokine as described above comprises IL-7. In some embodiments, the concentration of the IL-7 (e.g., human recombinant IL-7) is 100 to 5000 IU/mL, 200 to 2000 IU/mL, 500 to 1000 IU/mL, or 500 to 800 IU/mL. In specific embodiments, the concentration of the IL-7 is or is about 500 IU/mL, 600 IU/mL, 700 IU/mL, 800 IU/mL, 900 IU/mL, or 1000 IU/mL. In some embodiments, the concentration of the IL-7 is or is about 700 IU/mL.

In some embodiments, the cytokine as described above comprises IL-15. In some embodiments, the concentration of the IL-15 (e.g., human recombinant IL-15) is 0 to 500 IU/mL, 10 to 200 IU/mL, 20 to 100 IU/mL, 30 to 100 IU/mL, 40 to 80 IU/mL, or 40 to 60 IU/mL. In specific embodiments, the concentration of the IL-15 is or is about 25 IU/mL, 30 IU/mL, 40 IU/mL, 46 IU/mL, 50 IU/mL, 60 IU/mL, 70 IU/mL, 80 IU/mL, 90 IU/mL, or 100 IU/mL. In some embodiments, the concentration of the IL-15 is or is about 46 IU/mL.

In some embodiments, the supplement as described above comprises an amino acid. In some embodiments, the amino acid is selected from aspartic acid, glutamic acid, asparagine, serine, glutamine, histidine, glycine, threonine, arginine, alanine, tyrosine, cysteine, valine, methionine, norvaline, tryptophan, phenylalanine, isoleucine, lysine, hydroxyproline, sarcosine, proline, or a combination thereof. In some embodiments, the supplement comprises glutamine. In some embodiments, the composition comprises a synthetic amino acid. In some embodiments, the synthetic amino acid is capable of being converted into a free form of L-glutamine in a cell culture comprising cells. In some embodiments, the synthetic amino acid is a dipeptide. In some embodiments, the synthetic amino acid is L-alanyl-L-glutamine (GlutaMax).

In some embodiments, the concentration of the amino acid (e.g., L-alanyl-L-glutamine) is about 0.5 mM to 5 mM. In some embodiments, the concentration of the L-alanyl-L-glutamine is or is about 0.5 mM to 1 mM, 0.5 mM to 1.5 mM, 0.5 mM to 2 mM, 0.5 mM to 2.5 mM, 0.5 mM to 3 mM, 0.5 mM to 3.5 mM, 0.5 mM to 4 mM, 0.5 mM to 4.5 mM, 0.5 mM to 5 mM, 1 mM to 1.5 mM, 1 mM to 2 mM, 1 mM to 2.5 mM, 1 mM to 3 mM, 1 mM to 3.5 mM, 1 mM to 4 mM, 1 mM to 4.5 mM, 1 mM to 5 mM, 1.5 mM to 2 mM, 1.5 mM to 2.5 mM, 1.5 mM to 3 mM, 1.5 mM to 3.5 mM, 1.5 mM to 4 mM, 1.5 mM to 4.5 mM, 1.5 mM to 5 mM, 2 mM to 2.5 mM, 2 mM to 3 mM, 2 mM to 3.5 mM, 2 mM to 4 mM, 2 mM to 4.5 mM, 2 mM to 5 mM, 2.5 mM to 3 mM, 2.5 mM to 3.5 mM, 2.5 mM to 4 mM, 2.5 mM to 4.5 mM, 2.5 mM to 5 mM, 3 mM to 3.5 mM, 3 mM to 4 mM, 3 mM to 4.5 mM, 3 mM to 5 mM, 3.5 mM to 4 mM, 3.5 mM to 4.5 mM, 3.5 mM to 5 mM, 4 mM to 4.5 mM, 4 mM to 5 mM, or 4.5 mM to 5 mM. In some embodiments, the concentration of the L-alanyl-L-glutamine is or is about 0.5 mM, 1 mM, 1.5 mM, 2 mM, 2.5 mM, 3 mM, 3.5 mM, 4 mM, 4.5 mM, or 5 mM. In specific embodiments, the concentration of the L-alanyl-L-glutamine is 2 mM.

In some embodiments, the supplement as described above comprises an antioxidant. In some embodiments, the antioxidant comprises N-acetyl-L-cysteine (NAC), 2-mercaptoethanol, or D,L-α-tocopherol acetate, or derivatives or mixtures thereof. In some embodiments, the antioxidant is N-acetyl-L-cysteine.

In some embodiments, the concentration of the N-acetyl-L-cysteine is or is about 0.1 to 10 mg/mL, 0.2 to 10 mg/mL, 0.5 to 10 mg/mL, 0.1 to 5 mg/mL, 0.5 to 1 mg/mL, 0.2 to 5 mg/mL, 0.5 to 5 mg/mL, 0.5 to 2 mg/mL, or 0.6 to 1 mg/mL. In some embodiments, the concentration of the N-acetyl-L-cysteine is or is about 0.1 mg/mL, 0.2 mg/mL, 0.5 mg/mL, 0.6 mg/mL, 0.7 mg/mL, 0.8 mg/mL, 0.9 mg/mL, 1 mg/mL, 2 mg/mL, 3 mg/mL, 4 mg/mL, or 5 mg/mL. In some embodiments, the concentration of the N-acetyl-L-cysteine is or is about 0.8 mg/mL.

II-c. Multicomponent Composition

In some embodiments, the composition as described above is a multicomponent composition. Each component in the multicomponent composition (e.g., each independent composition or medium) may be used separately for cell culture. In some embodiments, the multicomponent composition comprises a plurality of independent media. In some embodiments, the multicomponent composition comprises a first medium, a second medium, a third medium, or any combination thereof. In some embodiments, the multicomponent composition comprises a first medium, a second medium, and a third medium.

In some embodiments, the first medium comprises the medium substrate according to any one of the preceding embodiments, and optionally a supplement. In some embodiments, the first medium is the medium substrate according to any one of the preceding embodiments. In some embodiments, the first medium comprises the medium substrate according to any one of the preceding embodiments, and optionally a supplement other than stimulants (e.g., cytokines). In some embodiments, the first medium comprises the medium substrate according to any one of the preceding embodiments, and optionally an amino acid and/or an antioxidant. The first medium may also be referred to as a basal medium. In some embodiments, the first medium may be used for cell culture before or after cell activation, such as cell isolation, washing, or expansion. In some embodiments, the first medium may be used in a step prior to cell activation.

In some embodiments, the second medium comprises the medium substrate according to any one of the preceding embodiments and a supplement. In some embodiments, the second medium comprises the medium substrate according to any one of the preceding embodiments and a cytokine. In some embodiments, the second medium comprises the medium substrate according to any one of the preceding embodiments, a cytokine, and optionally an amino acid and/or an antioxidant. In some embodiments, the second medium comprises the medium substrate according to any one of the preceding embodiments, a cytokine, an amino acid, and an antioxidant. The second medium may also be referred to as an activation medium and may be used in the step of cell activation.

In some embodiments, the third medium comprises the medium substrate according to any one of the preceding embodiments, and optionally a supplement. In some embodiments, the third medium comprises the medium substrate according to any one of the preceding embodiments, a cytokine, and optionally a cytokine and/or an amino acid. In some embodiments, the third medium comprises the medium substrate according to any one of the preceding embodiments, a cytokine, and an amino acid. The third medium may also be referred to as an expansion medium. In some embodiments, the third medium may be used in steps after cell activation, such as the step of cell expansion.

II-d. Preparation of Composition

In one aspect, the present disclosure provides a method for preparing a composition, comprising mixing the foregoing components simultaneously or sequentially. In some embodiments, the method comprises mixing a first serum-free medium and a second serum-free medium in a medium substrate, followed by the addition of a supplement, such as a cytokine, L-alanyl-L-glutamine, N-acetyl-L-cysteine, or a combination thereof.

### III. CELL CULTURE

In one aspect, the present disclosure provides a method for culturing cells, which employs the composition according to any one of the preceding embodiments or a combination thereof. In some embodiments, the cell is an immune cell, such as a T cell. In some embodiments, the cell is a genetically engineered cell, such as a genetically engineered T cell. In some embodiments, the cells are cultured for at least about 1, 2, 3, 4, 5, 6, 7, 8, 9, 10, 11, 12, 13, 14, 15, 16, 17, 18, 19, or 20 days. In some embodiments, the cells are cultured for about 2 to 3 days, 3 to 4 days, 4 to 5 days, 5 to 6 days, 6 to 7 days, 7 to 8 days, 8 to 9 days, 9 to 10 days, 10 to 11 days, 11 to 12 days, 12 to 13 days, 13 to 14 days, 14 to 15 days, 15 to 16 days, 16 to 17 days, 17 to 18 days, 18 to 19 days, or 19 to 20 days, each inclusive. In some embodiments, the cells are cultured at 37.0±1.0° C. In some embodiments, the cells are cultured under conditions of 5.0±1.0% CO₂. In some embodiments, the culture may be performed for greater than or greater than about 1, 2, 3, 4, 5, 6, 7, 8, 9, 10, 11, 12, 13, 14, 15, 16, 17, 18, 19, or 20 days without medium exchange during cell culture. In some embodiments, perfusion (e.g., semi-continuous perfusion) methods may be used in combination with cell culture.

In some embodiments, the cell is an immune cell or an enriched immune cell. In some embodiments, the cell is a T cell or an enriched T cell. In some embodiments, the cell is a CD4⁺ T cell or an enriched CD4⁺ T cell. In some embodiments, the cell is a CD8⁺ T cell or an enriched CD8⁺ T cell. In some embodiments, the cells are CD4⁺ T cells and CD8⁺ T cells. In some embodiments, the cells are enriched CD4⁺ T cells and enriched CD8⁺ T cells. In some embodiments, the cell comprises a genetically engineered cell or an enriched population of genetically engineered cells. In some embodiments, the cell comprises cells to be genetically engineered or being genetically engineered. In some embodiments, the cell comprises an enriched population of cells to be genetically engineered or being genetically engineered. In some embodiments, the cell comprises a genetically engineered T cell or an enriched population of genetically engineered T cells. In some embodiments, the cell comprises chimeric antigen receptor (CAR)-expressing T cells or enriched CAR-expressing T cells. In some embodiments, the cell comprises a T cell expressing a caTCR that binds AFP and a CSR that binds GPC-3 (e.g., Example 1 and Example 3). In some embodiments, the cell comprises a T cell expressing a caTCR that binds GPC-3 and a CSR that binds GPC-3 (e.g., Example 2). In some embodiments, the cells have been previously cryopreserved. In some embodiments, the cells have been cultured in serum-free medium and cryopreserved after culture. In some embodiments, the cells specifically target tumor cells.

In some embodiments, the cells are expanded at least or at least about 2-fold, 5-fold, 10-fold, 20-fold, 30-fold, 40-fold, 50-fold, 60-fold, 70-fold, 80-fold, 90-fold, 100-fold, 150-fold, 200-fold, 250-fold, 300-fold, 350-fold, 400-fold, 450-fold, 500-fold, or more after about 1, 2, 3, 4, 5, 6, 7, 8, 9, 10, 11, 12, 13, 14, 15, 16, 17, 18, 19, 20 days, or more of culture using the cell culture method of the present disclosure. In some embodiments, the cells are expanded at least 20-fold, 30-fold, 40-fold, 50-fold, 60-fold, 70-fold, 80-fold, 90-fold, 100-fold, or more after about 9 days of culture in a serum-free medium formulation. In some embodiments, the composition of the present disclosure exhibits a higher fold expansion of immune cells in cell culture compared to the first serum-free medium and the second serum-free medium alone.

In some embodiments, the cell culture method involves one or more steps or processes. In some embodiments, the one or more steps comprise isolation, selection, activation, transduction, incubation, expansion, washing, suspension, dilution, concentration, and/or formulation of cells. In some embodiments, the method comprises isolating cells before preparing, processing, and culturing the cells under one or more stimulatory conditions. In some embodiments, the method comprises processing steps in the following order: first isolating (e.g., selecting) cells from a biological sample; incubating the selected cells with viral vector particles for transduction, e.g., after the step of stimulating the isolated cells in the presence of a stimulant; and culturing the transduced cells, e.g., expanding the cells.

In some embodiments, the one or more processing steps may comprise one or more of: (a) pre-washing a biological sample containing cells (e.g., a whole blood sample, a buffy coat sample, a peripheral blood mononuclear cell (PBMC) sample, an unfractionated T cell sample, a lymphocyte sample, a leukocyte sample, an apheresis product, or a leukapheresis product); (b) isolating (e.g., selecting) desired cells (e.g., CD4⁺ and/or CD8⁺ T cells) from the sample, e.g., by incubating the cells with an immunoaffinity reagent; (c) introducing a vector encoding a recombinant receptor into the isolated or selected cells, e.g., by incubating the isolated (e.g., selected) cells with viral vector particles encoding the recombinant receptor; and (d) culturing or expanding the cells. In some embodiments, the method may further comprise (f) activating the cells by exposing the cells to stimulatory conditions, which may be performed before, during, and/or after incubating the cells with the viral vector particles, e.g., between steps (b) and (c). In some embodiments, the step (f) is performed prior to incubating the cells with the viral vector particles. In some embodiments, a washing and/or suspending step may also be performed before or after any one of the foregoing steps, e.g., between steps (b) and (f). In some embodiments, one or more or all of the steps of the cell culture method are performed under sterile conditions. In some embodiments of the method, cell isolation, transduction, washing, optionally activation or stimulation, and formulation are all performed in a closed system.

III-a. Cell Isolation

In some embodiments, the cell culture method comprises isolating cells or a composition thereof from a biological sample. In some embodiments, the biological sample is blood or a sample derived from blood, or is or is derived from an apheresis or leukapheresis product. Exemplary samples include whole blood, peripheral blood mononuclear cells (PBMCs), leukocytes, bone marrow, thymus, tissue biopsy, tumors, leukemia, lymphoma, lymph nodes, gut-associated lymphoid tissue, mucosa-associated lymphoid tissue, spleen, liver, lung, stomach, intestine, colon, kidney, pancreas, breast, bone, prostate, cervix, testis, ovary, tonsils, or other organs and/or cells derived therefrom. In some embodiments, biological samples include samples from autologous and allogeneic sources.

In some embodiments, the cell is a mammalian cell or a human cell. In some embodiments, the cell is an immune cell, such as an innate or adaptive immune cell, e.g., a myeloid or lymphoid cell (including lymphocytes, e.g., T cells and/or NK cells). In some embodiments, the cell is a primary cell. In some embodiments, the cells are allogeneic and/or autologous. In some embodiments, the cells comprise one or more subsets of T cells or other cell types, such as the entire T cell population, CD4⁺ cells, CD8⁺ cells, and subpopulations thereof. Subtypes and subpopulations of T cells and/or CD4⁺ and/or CD8⁺ T cell include naive T (T_{N}) cells, effector T cells (T_{EFF}), memory T cells and subtypes thereof (e.g., stem cell memory T (T_{SCM}), central memory T (T_{CM}), effector memory T (T_{EM}), or terminally differentiated effector memory T cells), tumor-infiltrating lymphocytes (TILs), immature T cells, mature T cells, helper T cells, cytotoxic T cells, mucosa-associated invariant T (MAIT) cells, naturally occurring and adaptive regulatory T (Treg) cells, helper T cells (e.g., TH1 cells, TH2 cells, TH3 cells, TH17 cells, TH9 cells, TH22 cells, follicular helper T cells), α/β T cells, and δ/γ T cells. In some embodiments, the cell is a natural killer (NK) cell. In some embodiments, the cell is a monocyte or granulocyte, such as a myeloid cell, a macrophage, a neutrophil, a dendritic cell, a mast cell, an eosinophil, and/or a basophil. In some embodiments, the cell is a tumor-infiltrating lymphocyte.

In some embodiments, the cell isolation step is non-affinity-based. In some examples, cells are washed and/or centrifuged in the presence of one or more reagents to remove unwanted components, enrich for desired components, lyse or remove cells sensitive to particular reagents. In some examples, cells are isolated based on one or more characteristics (e.g., density, adhesion properties, size, sensitivity and/or resistance to particular components). In some embodiments, the method includes a density-based cell isolation method, e.g., lysing red blood cells and preparing leukocytes from peripheral blood by gradient centrifugation.

In some embodiments, the cell isolation step comprises incubating the cells with a selection agent, followed by isolation. In some embodiments, the selection agent is an immunoaffinity-based cell-binding agent. In some embodiments, the selection agent is an antibody or a conjugate thereof that specifically binds to a cell marker. In some embodiments, the selection agent is a particle (e.g., a magnetic bead) conjugated with a specific selection agent (e.g., an antibody). In some embodiments, the magnetic bead comprises a magnetically responsive material conjugated to an antibody. In some embodiments, the magnetically responsive material is coated with a primary antibody (a cell-binding antibody), a secondary antibody, a lectin, an enzyme, or streptavidin, and attached to the cell via the primary antibody. The cell isolation step comprises isolating cells based on the expression or expression level of one or more markers (typically cell surface markers) in the cells, e.g., by incubating the cells with an antibody or a conjugate thereof that specifically binds to the marker, followed by washing and isolation to obtain cells bound to the antibody (positive selection) or cells not bound to the antibody (negative selection). In some embodiments, the cell isolation step comprises enriching a particular cell population via positive selection or depleting a particular cell population via negative selection. In some embodiments, the positive or negative selection is accomplished by incubating the cells with one or more antibodies or other binding agents that specifically bind to one or more surface markers expressed or expressed at relatively high levels on the positively or negatively selected cells, respectively. The isolation does not require 100% enrichment or removal of a particular cell population or cells expressing a particular marker. In some embodiments, the step comprises multiple rounds, e.g., subjecting the positively or negatively selected cells to another positive or negative selection. Specific subpopulations of T cells, such as cells positive for or exhibiting high-level expression of one or more surface markers (e.g., CD28⁺, CD62L⁺, CCR7⁺, CD27⁺, CD127⁺, CD4⁺, CD8⁺, CD45RA⁺, and/or CD45RO⁺ T cells), may be isolated by positive or negative selection techniques. In some embodiments, such cells are selected by incubation with one or more antibodies or conjugates thereof that specifically bind to such markers. For example, CD4⁺ and CD8⁺ T cells may be positively selected using magnetic beads conjugated to anti-CD4 antibodies and anti-CD8 antibodies.

In some embodiments, the cell isolation step is performed in a buffer. In some embodiments, the buffer and selection agent are mixed prior to adding the cells. In some embodiments, the selection buffer and selection agent are separately added to the cell sample. In some embodiments, the total duration of incubation with the selection agent is from or from about 5 minutes to or to about 6 hours, such as 30 minutes to 3 hours, e.g., at least or at least about 30 minutes, 60 minutes, 120 minutes, or 180 minutes.

In some embodiments, the cell isolation step further comprises pre-washing a cell-containing sample (e.g., an apheresis sample). In some embodiments, the immunoaffinity-based isolation system is or contains a magnetic separation column. The isolation step comprises placing the sample in a magnetic field, wherein cells bound to magnetic beads will be attracted to the magnetic separation column and separated from cells not bound to magnetic beads. For positive selection, cells attracted to the magnet are retained; for negative selection, cells not attracted are retained. In some embodiments, affinity-based selection is performed via magnetic-activated cell sorting (MACS, e.g., CliniMACS system, Miltenyi Biotec). In some embodiments, the step is performed in the inner cavity of a centrifugal chamber, e.g., under centrifugal rotation. In some embodiments, the steps are performed in an automated manner, e.g., the steps of pre-washing, binding, and isolation are accomplished in a single closed system using an automated procedure.

In some embodiments, the cell isolation step comprises a step of freezing (e.g., cryopreserving) the cells after isolation. In some embodiments, the freezing step comprises removing granulocytes and, to some extent, monocytes from the cell population. In some embodiments, the cells are frozen at a rate of 1° C./min to −80° C. for storage.

III-b. Cell Activation

In some embodiments, the step comprises a step of stimulating isolated cells (e.g., a selected population of cells). In some embodiments, cell activation is performed prior to or simultaneously with genetic engineering (e.g., transduction). In some embodiments, cell activation is performed prior to genetic engineering.

In some embodiments, compositions or cells are incubated under stimulatory and/or activation conditions. In some embodiments, conditions for stimulation and/or activation may comprise one or more of: a specific medium, temperature, oxygen content, carbon dioxide content, time, drugs (e.g., nutrients, amino acids, antibiotics, ions, and/or stimulants such as cytokines, chemokines, antigens, fusion proteins, recombinant soluble receptors, and any other agents capable of activating cells). In some embodiments, the stimulatory conditions include one or more drugs capable of stimulating or activating an intracellular signaling domain of a TCR complex. In some embodiments, the drug is capable of initiating a TCR/CD3 intracellular signaling cascade in T cells, e.g., a drug (e.g., an anti-CD3 antibody) suitable for delivering a primary signal to, for example, initiate activation of an ITAM-induced signal, and/or an agent that promotes a co-stimulatory signal (e.g., a co-stimulatory signal specific for a T cell co-stimulatory receptor), such as an anti-CD28 or anti-4-1BB antibody (e.g., bound to a solid support) and/or one or more cytokines. The stimulant includes an anti-CD3 antibody and an anti-CD28 antibody, or magnetic beads conjugated to anti-CD3/anti-CD28 antibodies (e.g., Dynabead™ CD3/CD28 T Cell Expander). In some embodiments, the stimulant comprises IL-2, IL-7, and/or IL-15. In some embodiments, the stimulatory conditions include a temperature suitable for the growth of human T lymphocytes, e.g., at least about 25° C., at least about 30° C., or about 37° C.

In some embodiments, the cell activation step is performed in the inner cavity of a centrifugal chamber. In some embodiments, the cells are mixed with stimulatory conditions or stimulants in a centrifugal chamber. In some embodiments, the cells are incubated in an activation medium comprising cytokines. In some embodiments, the volume of the activation medium is 10 mL to 200 mL, or about 10 mL, 20 mL, 30 mL, 40 mL, 50 mL, 60 mL, 70 mL, 80 mL, 90 mL, 100 mL, 150 mL, or 200 mL. In some embodiments, the activation medium comprises a medium substrate and a supplement. In some embodiments, the supplement comprises a cytokine, and optionally an amino acid and an antioxidant. In some embodiments, the cytokine is IL-2, IL-7, and IL-15. In some embodiments, the activation medium is pre-formulated. In some embodiments, the medium substrate and supplement are separately added to the cells.

In some embodiments, the total duration of the activation process is between or between about 1 hour and 96 hours, 1 hour and 72 hours, 1 hour and 48 hours, 4 hours and 36 hours, 8 hours and 30 hours, or 12 hours and 24 hours, e.g., at least or at least about 6 hours, 12 hours, 18 hours, 24 hours, 30 hours, 36 hours, or 72 hours.

III-c. Genetic Engineering

In one aspect, the cell culture method comprises a step of genetic engineering. In some embodiments, the step comprises introducing a nucleic acid molecule encoding a recombinant protein into a cell. In some embodiments, the nucleic acid molecule encoding the recombinant protein is introduced into the cell before, simultaneously with, or after cell activation. In some embodiments, the nucleic acid molecule encoding the recombinant protein is introduced into the cell after cell activation. In some embodiments, the introduction is performed by contacting the cell with the nucleic acid molecule encoding the recombinant protein. The step is optional for the cell culture method of the present disclosure.

In some embodiments, the genetic engineering step comprises introducing the recombinant protein into the cell via a vector. Such vectors include viral and non-viral systems. In some embodiments, the viral system comprises recombinant infectious viral particles, such as vectors derived from adenovirus, adeno-associated virus (AAV), and human immunodeficiency virus (HIV), as well as recombinant lentiviral vectors or retroviral vectors (e.g., gamma retroviral vectors). In some embodiments, the non-viral system comprises a transposon system, such as a PiggyBac or Sleeping Beauty gene transfer system. In some embodiments, the step is performed by electroporation. In some embodiments, the step is performed by transduction, transposon, electroporation, or a combination thereof. Other methods for introducing and expressing genetic material in immune cells include calcium phosphate transfection, protoplast fusion, cationic liposome-mediated transfection, tungsten particle-facilitated microprojectile bombardment, and strontium phosphate DNA co-precipitation.

In some embodiments, the concentration of cells to be transduced ranges from or from about 1.0×10⁵ cells/mL to 1.0×10⁸ cells/mL, such as at least or at least about or about 1.0×10⁵ cells/mL, 5×10⁵ cells/mL, 1×10⁶ cells/mL, 5×10⁶ cells/mL, 1×10⁷ cells/mL, 5×10⁷ cells/mL, or 1×10⁸ cells/mL. In some embodiments, the viral particles are provided at a ratio of viral vector particle copies or infectious units thereof (IU) to the total number of cells to be transduced (IU/cell). In some embodiments, the viral particles are present at or at least 0.5, 1, 2, 3, 4, 5, 10, 15, 20, 30, 40, 50, or 60 IU of viral vector particles per cell. In some embodiments, transduction may be achieved at a multiplicity of infection (MOI) of less than 100, e.g., typically less than 60, 50, 40, 30, 20, 10, 5, or less.

In some embodiments, the step comprises contacting a cell with a viral particle. In some embodiments, the contacting lasts for a period of 30 minutes to 72 hours, 30 minutes to 48 hours, 30 minutes to 24 hours, or 1 hour to 24 hours, or at least about 30 minutes, 1 hour, 2 hours, 6 hours, 12 hours, 24 hours, or 36 hours. In some embodiments, the contacting is performed in a solution or medium. In some embodiments, the contacting is performed in a second medium. In some embodiments, the cells and viral particles are contacted in a volume of 0.5 mL to 500 mL, e.g., the volume being or being about 0.5 mL to 200 mL, 0.5 mL to 100 mL, 0.5 mL to 50 mL, 0.5 mL to 10 mL, 0.5 mL to 5 mL, 5 mL to 500 mL, 5 mL to 200 mL, 5 mL to 100 mL, 5 mL to 50 mL, 5 mL to 10 mL, 10 mL to 500 mL, 10 mL to 200 mL, 10 mL to 100 mL, 10 mL to 50 mL, 50 mL to 500 mL, 50 mL to 200 mL, 50 mL to 100 mL, 100 mL to 500 mL, 100 mL to 200 mL, or 200 mL to 500 mL. In some embodiments, the contacting is achieved by centrifugation. In some embodiments, the centrifugation is at a speed lower than that used for pelleting cells, e.g., from or from about 600 rpm to or to about 1700 rpm, e.g., at a speed of or about or at least 600 rpm, 1000 rpm, 1500 rpm, or 1700 rpm.

In some embodiments, after the genetic engineering step, the cells are transferred to another vessel for culturing the genetically engineered cells, e.g., for cell expansion. In some embodiments, the vessel for cell expansion is a bioreactor bag, such as a perfusion bag.

III-d. Cell Expansion

In one aspect, the cell culture method comprises a step of expanding cells, which is performed in an expansion medium. In some embodiments, the cells are expanded following the genetic engineering step. In some embodiments, the expansion medium is any one of the compositions as described in II. Medium. The composition has a surprising cell expansion effect.

In some embodiments, the expansion step lasts for a period greater than or equal to 24 hours, 48 hours, 72 hours, 96 hours, 5 days, 6 days, 7 days, 8 days, or 9 days. In some embodiments, the expansion step is performed at a temperature of at least about 25° C., at least about 30° C., or about 37° C. In some embodiments, the temperature is 25 to 38° C. or 36 to 38° C. In some embodiments, the cells are expanded under conditions that maintain a target amount of carbon dioxide (CO₂) in the cell culture. In some embodiments, the amount of CO₂ is 10% to 0% (v/v), 8% to 2% (v/v), or is or is about 5% (v/v) of the culture gas.

In some embodiments, the cells are expanded using a vessel in combination with a bioreactor. In some embodiments, the bioreactor is subjected to movement or rocking. Moving the bioreactor may increase oxygen transfer, including, but not limited to, rotation along a horizontal axis, rotation along a vertical axis, rocking motion along an inclined horizontal axis of the bioreactor, or any combination thereof. In some embodiments, the rocking angle is or is about 20°, 19°, 18°, 17°, 16°, 15°, 14°, 13°, 12°, 11°, 10°, 9°, 8°, 7°, 6°, 5°, 4°, 3°, 2°, or 1°. In certain embodiments, the rocking angle is 6° to 16°. In other embodiments, the rocking angle is 7° to 16°. In other embodiments, the rocking angle is 8° to 12°. In some embodiments, the rocking angle is 5° to 10°, such as 6°. In some embodiments, the rocking rate is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 rpm. In some embodiments, the rocking rate is 4 rpm to 12 rpm, such as 4 rpm to 6 rpm, inclusive. In some embodiments, the constant rocking rate is 5 rpm to 15 rpm, such as 6 rpm or 10 rpm. In some embodiments, the bioreactor is maintained at a temperature equal to or near 37° C. and a CO₂ level equal to or near 5%, with a steady air flow rate of or at least 0.01 L/min, 0.05 L/min, 0.1 L/min, 0.2 L/min, 0.3 L/min, 0.4 L/min, 0.5 L/min, 1.0 L/min, 1.5 L/min, or 2.0 L/min or greater. In certain embodiments, expansion is performed under perfusion.

In some embodiments, the expansion step is performed in a closed system. In some embodiments, the expansion step is performed in the same closed system as one or more other steps of the cell culture method.

### IV. RECOMBINANT PROTEIN

In one aspect, cells cultured with compositions of the present disclosure express recombinant proteins. In some embodiments, the cell comprises one or more nucleic acids encoding a recombinant protein introduced by genetic engineering, thereby expressing the recombinant protein. In some embodiments, expression of the recombinant protein is achieved by first activating cells, then transducing the activated cells, and expanding them in medium to a quantity sufficient for clinical application.

In some embodiments, the recombinant protein is a chimeric receptor, a chimeric antigen receptor (CAR), a T cell receptor (TCR), a chimeric antibody-T cell construct (caTCR), a chimeric signaling receptor (CSR), or a combination thereof.

IV-a. Chimeric Antigen Receptor (CAR)

In some embodiments, the recombinant protein is a chimeric antigen receptor (CAR). In some embodiments, the antigen is selectively expressed or overexpressed on cells of a disease or disorder (e.g., tumor or pathogenic cells) compared to normal or non-target cells. In some embodiments, the diseases and disorders comprise proliferative, neoplastic, and malignant diseases and disorders, including cancers and tumors, including hematologic cancer, cancer of the immune system, such as lymphoma, leukemia, and/or myeloma, e.g., B-cell leukemia, T-cell leukemia, myeloid leukemia, lymphoma, and multiple myeloma. In some embodiments, the antigen is a tumor antigen or a cancer marker. In some embodiments, the antigen (including a ligand) is or comprises B-cell maturation antigen (BCMA), B7-H3, B7-H6, carbonic anhydrase 9 (CA9), cancer-testis antigen, cancer/testis antigen 1B (NY-ESO-1), carcinoembryonic antigen (CEA), cyclin, cyclin A2, C—C motif chemokine ligand 1 (CCL-1), CD19, CD20, CD22, CD23, CD24, CD30, CD33, CD38, CD44, CD44v6, CD44v7/8, CD123, CD133, CD138, CD171, MAGE-A4, DLL3, CEA, Claudin18.2 (CLDN18.2), chondroitin sulfate proteoglycan 4 (CSPG4), epidermal growth factor protein (EGFR), epidermal growth factor receptor variant III (EGFR vIII), epithelial glycoprotein 2 (EPG-2), epithelial glycoprotein 40 (EPG-40), ephrin B2, ephrin receptor A2 (EPHa2), estrogen receptor, Fc receptor-like protein 5 (FCRL5), fetal acetylcholine receptor (fetal AchR), folate-binding protein (FBP), folate receptor α, ganglioside GD2, O-acetylated GD2 (OGD2), ganglioside GD3, glycoprotein 100 (gp100), glypican-3 (GPC3), G protein-coupled receptor class C group 5 member D (GPRC5D), GUCY2C, Her2/neu (receptor tyrosine kinase erb-B2), Her3 (erb-B3), Her4 (erb-B4), erbB dimer, human high molecular weight melanoma-associated antigen (HMW-MAA), hepatitis B surface antigen, human leukocyte antigen A1 (HLA-A1), human leukocyte antigen A2 (HLA-A2), IL-22 receptor α (IL-22Rα), IL-13 receptor α2 (IL-13Rα2), kinase insert domain receptor (kdr), K light chain, L1 cell adhesion molecule (L1-CAM), CE7 epitope of L1-CAM, leucine-rich repeat-containing 8 family member A (LRRC8A), Lewis Y, melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligand, Melan-A (MART-1), neural cell adhesion molecule (NCAM), carcinoembryonic antigen, preferentially expressed antigen in melanoma (PRAME), progesterone receptor, prostate-specific antigen, prostate stem cell antigen (PSCA), prostate-specific membrane antigen (PSMA), receptor tyrosine kinase-like orphan receptor 1 (ROR1), survivin, trophoblast glycoprotein (TPBG, also known as 5T4), tumor-associated glycoprotein 72 (TAG72), tyrosinase-related protein 1 (TRP1, also known as TYRP1 or gp75), tyrosinase-related protein 2 (TRP2, also known as dopachrome tautomerase, dopachrome 8-isomerase, or DCT), vascular endothelial growth factor receptor (VEGFR), vascular endothelial growth factor receptor 2 (VEGFR2), Wilms tumor 1 (WT-1), pathogen-specific or pathogen-expressed antigens, or antigens associated with universal tags, and/or biotinylated molecules, and/or molecules expressed by HIV, HCV, HBV, or other pathogens. In some embodiments, the receptor-targeted antigen comprises an antigen associated with a B-cell malignancy, such as any of a variety of known B-cell markers. In some embodiments, the antigen is or comprises CD20, CD19, CD22, ROR1, CD45, CD21, CD5, CD33, IgK, Ig), CD79a, CD79b, or CD30.

In some embodiments, the CAR comprises an extracellular antigen recognition domain that specifically binds to an antigen. Therefore, the extracellular antigen recognition domain that specifically binds to the antigen comprises one or more antigen-binding molecules, such as one or more antigen-binding fragments, domains, or portions, or one or more antibody variable domains, and/or antibodies. In some embodiments, the antigen-binding molecule is a full-length antibody, e.g., a single-domain antibody, a monospecific antibody, or a multispecific antibody; or an antibody fragment, e.g., Fv, Fab, Fab′, Fab′-SH, F(ab′)₂, or scFv.

In some embodiments, the CAR comprises a spacer, which may be or comprise at least a portion of an immunoglobulin constant region or a variant or modified form thereof, such as a hinge region and/or a CHI/CL and/or an Fc region. In some embodiments, the spacer is located between the extracellular antigen recognition domain and the transmembrane domain.

In some embodiments, the CAR comprises a transmembrane domain. In some embodiments, the transmembrane domain is derived from a natural source or from a synthetic source. Where the source is natural, the transmembrane domain is derived from any membrane-binding protein or transmembrane protein. The transmembrane domain comprises one or more of the α, β, or ζ chains of T cell receptors, CD28, CD3ε, CD45, CD4, CD5, CD8, CD9, CD16, CD22, CD33, CD37, CD64, CD80, CD86, CD134, CD137, or CD154. In some embodiments, the transmembrane domain is synthetic. In some aspects, the synthetic transmembrane domain primarily comprises hydrophobic amino acid residues, such as leucine and valine.

In some embodiments, the CAR comprises an intracellular signaling domain, wherein the intracellular signaling domain comprises a cytoplasmic signaling domain, e.g., an intracellular domain capable of inducing a primary activation signal in a T cell, such as the ζ chain of the CD3ζ chain; and/or the intracellular signaling domain comprises an immunoreceptor tyrosine-based activation motif (ITAM). In some embodiments, the CAR comprises a co-stimulatory domain. In some embodiments, the co-stimulatory domain is a signaling region and/or transmembrane portion of CD28, 4-1BB, OX40, DAP10, or ICOS. In some aspects, the same CAR comprises both a primary activation signaling region and a co-stimulatory component. In some embodiments, the same CAR comprises both a primary activation signaling domain and a co-stimulatory domain.

IV-b. T Cell Receptor (TCR)

In some embodiments, the recombinant protein is a TCR. In some embodiments, the TCR is naturally occurring or modified. In some embodiments, the TCR comprises variable α and β chains (TCR α and TCR β) or variable γ and δ chains (TCR γ and TCR δ) or antigen-binding fragments thereof, and is capable of specifically binding to a peptide bound to an MHC molecule. In some embodiments, the TCR may be a heterodimer of two chains α and β, or the TCR may be a single-chain TCR construct. In some embodiments, the TCR is a heterodimer comprising two separate chains linked, e.g., by one or more disulfide bonds. In some embodiments, the TCR further comprises a constant domain, a transmembrane domain, and/or a short cytoplasmic tail. In some embodiments, the TCR chain comprises one or more constant domains, e.g., the extracellular domain of the TCR chain comprises two immunoglobulin-like domains adjacent to the cell membrane, such as a variable domain and a constant domain. In some embodiments, the extracellular domain of the TCR formed by two chains comprises two membrane-proximal constant domains and two membrane-distal variable domains. The constant domain of the TCR may contain a short linker sequence in which cysteine residues form a disulfide bond, thereby linking the two chains of the TCR. In some embodiments, the TCR may have additional cysteine residues in each of the α and β chains, such that the TCR contains two disulfide bonds in the constant domain.

IV-c. Chimeric Antibody-T Cell Construct (caTCR)

In some embodiments, the recombinant protein is a chimeric antibody-T cell construct (caTCR). In some embodiments, the caTCR comprises a naturally occurring TCR domain or at least one non-naturally occurring TCR domain. In some embodiments, the caTCR comprises an antigen-binding module that provides antigen specificity and a T cell receptor module (TCRM) that enables CD3 recruitment and signaling. In some embodiments, the antigen-binding module is attached to the amino terminus of the TCRM. In some embodiments, the antigen-binding module is an antigen-binding molecule, including a full-length antibody, e.g., a single-domain antibody, a monospecific antibody, or a multispecific antibody; or an antibody fragment, e.g., Fv, Fab, Fab′, Fab′-SH, F(ab′)₂, or scFv. In some embodiments, the TCRM comprises a transmembrane module (TCR-TM) derived from the transmembrane domains of one or more TCRs, such as αβ and/or γδ TCRs, and optionally further comprises one or both of a linker peptide of the TCR or a fragment thereof and/or one or more TCR intracellular domains or fragments thereof.

In some embodiments, the TCRM comprises two polypeptide chains, each of which comprises, from the amino terminus to the carboxyl terminus, a linker peptide, a transmembrane domain, and optionally a TCR intracellular domain. In some embodiments, the TCRM comprises one or more non-naturally occurring TCR domains. In some embodiments, the caTCR may comprise a first polypeptide chain and a second polypeptide chain, wherein the first and second polypeptide chains together form an antigen-binding module and a TCRM. In some embodiments, the first and second polypeptide chains are separate polypeptide chains, and the caTCR is a multimer, such as a dimer. In some embodiments, the first and second polypeptide chains are covalently linked, e.g., via a peptide bond or via another chemical bond (e.g., a disulfide bond). In some embodiments, the first polypeptide chain is linked to the second polypeptide chain via at least one disulfide bond.

In some embodiments, the caTCR further comprises one or more T cell co-stimulatory signaling sequences. The one or more co-stimulatory signaling sequences may individually be all or a portion of an intracellular domain of a co-stimulatory molecule, including, for example, CD27, CD28, 4-1BB, OX40, CD30, CD40, ICOS, lymphocyte function-associated antigen-1 (LFA-1), CD2, CD7, LIGHT, NKG2C, B7-H3, ligands that specifically bind to CD83, and analogs thereof. In some embodiments, the one or more co-stimulatory signaling sequences are located between the first TCR-TM and the first TCR intracellular domain and/or between the second TCR-TM and the second TCR intracellular domain. In some embodiments, the one or more co-stimulatory signaling sequences are located at the carboxyl terminus of the first TCR intracellular domain and/or the second TCR intracellular domain. In some embodiments, the caTCR lacks a T cell co-stimulatory signaling sequence.

In some embodiments, the caTCR further comprises a stabilization module comprising a first stabilization domain and a second stabilization domain, wherein the first and second stabilization domains have binding affinity to each other that stabilizes the caTCR. In some embodiments, the stabilization module is located between the antigen-binding module and the TCRM. In some embodiments, the caTCR further comprises a spacer module between any two caTCR modules or domains. In some embodiments, the spacer module comprises one or more peptide linkers connecting two caTCR modules or domains.

IV-d. Chimeric Signaling Receptor (CSR)

In some embodiments, the recombinant protein is a chimeric co-stimulatory receptor (CSR). In some embodiments, the CSR comprises a ligand-binding module that provides ligand-binding specificity, a transmembrane module, and a co-stimulatory immune cell signaling module. In some embodiments, the CSR lacks a primary activation signaling sequence. In some embodiments, the CSR comprises a single polypeptide chain comprising a ligand-binding module, a transmembrane module, and a co-stimulatory signaling module. In some embodiments, the CSR comprises a first polypeptide chain and a second polypeptide chain, wherein the first and second polypeptide chains together form a ligand-binding module, a transmembrane module, and a co-stimulatory signaling module. In some embodiments, the first and second polypeptide chains are separate polypeptide chains, and the CSR is a multimer, such as a dimer. In some embodiments, the first and second polypeptide chains are covalently linked, e.g., via a peptide bond or via another chemical bond (e.g., a disulfide bond). In some embodiments, the first polypeptide chain is linked to the second polypeptide chain via at least one disulfide bond.

In some embodiments, the target ligand is a cell surface antigen. In some embodiments, the target ligand is a peptide/MHC complex. In some embodiments, the target ligand is the same as the target antigen of the caTCR expressed in the same immune cell. In some embodiments, the target ligand is different from the target antigen of the caTCR expressed in the same immune cell. In some embodiments, the target ligand is a molecule presented on the surface of a cell presenting the target antigen. In some embodiments, the target ligand is a disease-associated ligand. In some embodiments, the target ligand is a cancer-associated ligand. In some embodiments, the cancer-associated ligand is CD19, CD20, CD22, CD47, IL4, GPC-3, ROR1, ROR2, BCMA, GPRC5D, or FCRL5. In some embodiments, the cancer-associated ligand is a peptide/MHC complex, which comprises peptides derived from proteins comprising WT-1, AFP, HPV16-E7, NY-ESO-1, PRAME, EBV-LMP2A, and PSA. In some embodiments, the target ligand is a virus-associated ligand. In some embodiments, the target ligand is an immune checkpoint molecule. In some embodiments, the immune checkpoint molecule comprises PD-L1, PD-L2, CD80, CD86, ICOSL, B7-H3, B7-H4, HVEM, 4-1BBL, OX40L, CD70, CD40, and GAL9. In some embodiments, the target ligand is an apoptotic molecule. In some embodiments, the apoptotic molecule comprises FasL, FasR, TNFR1, and TNFR2.

In some embodiments, the transmembrane module comprises one or more transmembrane domains derived from, e.g., CD28, CD38, CD35, CD45, CD4, CD5, CD8, CD9, CD16, CD22, CD33, CD37, CD64, CD80, CD86, CD134, CD137, or CD154.

In some embodiments, the co-stimulatory immune cell signaling module is a portion of an intracellular domain of a co-stimulatory molecule, wherein the co-stimulatory molecule comprises CD27, CD28, 4-1BB, OX40, CD30, CD40, PD-1, ICOS, LFA-1, CD2, CD7, LIGHT, NKG2C, B7-H3, ligands that specifically bind to CD83, and analogs thereof.

In particular embodiments, the recombinant protein of the present disclosure comprises CAR, TCR, or both caTCR and CSR as described above. Those skilled in the art will appreciate that the medium of the present disclosure has the effect of enhancing cell expansion and is suitable for cells that have not been transduced or have been transduced with any of the recombinant proteins.

### V. EXAMPLES

### Example 1: Effect of Compositions on T Cell Culture

Human leukapheresis products were washed by centrifugation, followed by incubation with anti-CD4 magnetic beads and CD8 magnetic beads (Miltenyi Biotec, Cat. No. 276-01 and Cat. No. 275-01, the same below). The incubated products were sorted using a magnetic sorting system (Miltenyi Biotec, model CliniMACS Plus instrument, the same below), and the sorted T cells were subsequently cryopreserved. T cells from two different donors (Donor 1 and Donor 2) were thawed, grouped after staining and counting, and washed twice by centrifugation in Medium-1. T cell pellet was resuspended in Medium-1, and the cells were assayed for viability, proportion of CD25⁺CD69⁺ T cells, and proportion of T_{SCM} (recorded as Day 0). T cells were added to Medium-2 at a cell density of no less than 2.5×10⁶ cells/mL, and activated using magnetic beads conjugated to anti-CD3 antibody and anti-CD28 antibody. The cells were then incubated in an incubator at 37.0±1.0° C. with 5.0±1.0% CO₂ for 24±6 hours.

Activated T cells were assayed for density, viability, proportion of CD25⁺CD69⁺ T cells, and proportion of T_{SCM}. Transduced T cells were supplemented with Medium-3 to the same volume per group. The cells were transferred to an incubator at 37.0±1.0° C. with 5.0±1.0% CO₂ for incubation, and T cells were assayed and supplemented with Medium-3 at the corresponding time points. Starting from Day 5, cells were passaged with a cell density of approximately 0.625×10⁶ cells/mL per passage.

The Medium-1, Medium-2, and Medium-3 were prepared according to Table 1. Specifically, the medium substrate of Medium-2 and Medium-3 was Medium-1 of the corresponding group, and the concentration of cytokines, GlutaMAX, or NAC was measured based on the volume of Medium-1 before the addition of cytokines, GlutaMAX, and NAC.

TABLE 1
Medium formula

| | | Medium-2 | | | | | |
|---|---|---|---|---|---|---|---|
| | | Medium-3 | | | | | |
| | | Medium-1 | IL-2 | IL-7 | IL-15 | GlutaMAX | NAC |
| No | Donor | (v:v) | (IU/mL) | (U/mL) | (U/mL) | (mM) | (mg/mL) |
| 1-1 | 1 | TexMACS | 100 | 4400 | 46 | 2 | 0.8 |
| 1-2 | 1 | X-VIVO 15 | 100 | 4400 | 46 | 2 | 0.8 |
| 1-3 | 1 | T + X (1:1) | 0 | 4400 | 46 | 0 | 0 |
| 2-1 | 2 | TexMACS | 0 | 4400 | 46 | 0 | 0 |
| 2-2 | 2 | TexMACS | 100 | 4400 | 46 | 2 | 0.8 |
| 2-3 | 2 | X-VIVO 15 | 100 | 4400 | 46 | 2 | 0.8 |
| 2-4 | 2 | T + X (1:1) | 0 | 4400 | 46 | 0 | 0 |

Note:
T + X (1:1) refers to a mixture of TexMACS and X-VIVO 15 at a volume ratio of 1:1, the same below.

The parameters of cumulative fold expansion, viability, activation state, and differentiation phenotype during T cell culture are shown in the table below.

TABLE 2
Cumulative fold expansion

| No | Day 0 | Day 1 | Day 3 | Day 5 | Day 7 | Day 9 | Day 11 | Day 13 |
|---|---|---|---|---|---|---|---|---|
| 1-1 | 1.00 | 0.61 | 1.08 | 4.62 | 13.45 | 37.39 | 92.85 | 202.74 |
| 1-2 | 1.00 | 0.30 | 0.53 | 1.07 | 0.10 | NA | NA | NA |
| 1-3 | 1.00 | 0.56 | 0.88 | 5.55 | 21.01 | 96.49 | 294.23 | 453.93 |
| 2-1 | 1.00 | 0.69 | 0.71 | 3.26 | 10.65 | 23.22 | 41.61 | 38.51 |
| 2-2 | 1.00 | 0.67 | 0.97 | 3.34 | 9.55 | 33.05 | 82.91 | 102.95 |
| 2-3 | 1.00 | 0.53 | 0.89 | 1.38 | 1.74 | 3.55 | 7.16 | 14.09 |
| 2-4 | 1.00 | 0.64 | 0.85 | 3.90 | 13.89 | 47.05 | 122.73 | 188.27 |

Note:
NA stands for cell death.

Although the T+X medium in this example did not contain stimulants or nutrients such as IL-2, GlutaMAX, or NAC, the cumulative fold expansion of both donor cells cultured in the T+X medium was significantly higher than that of cells in other groups, demonstrating a surprising synergistic effect.

TABLE 3
Cell viability (%)

| No | Day 0 | Day 1 | Day 3 | Day 5 | Day 7 | Day 9 | Day 11 | Day 13 |
|---|---|---|---|---|---|---|---|---|
| 1-1 | 81.90 | 77.07 | 86.23 | 89.90 | 91.23 | 90.37 | 87.00 | 83.10 |
| 1-2 | 80.90 | 42.80 | 71.23 | 56.77 | 6.40 | NA | NA | NA |
| 1-3 | 82.37 | 58.27 | 69.13 | 88.07 | 92.07 | 93.50 | 92.40 | 90.87 |
| 2-1 | 84.53 | 77.70 | 83.10 | 92.03 | 94.07 | 94.10 | 90.17 | 68.20 |
| 2-2 | 84.53 | 79.97 | 90.17 | 91.23 | 94.00 | 91.83 | 86.97 | 73.50 |
| 2-3 | 82.40 | 63.10 | 77.50 | 69.50 | 62.53 | 73.10 | 78.73 | 85.53 |
| 2-4 | 84.10 | 68.97 | 77.00 | 87.30 | 92.07 | 93.67 | 93.57 | 89.43 |

Note:
NA stands for cell death.

Although the T+X medium in this example did not contain stimulants or nutrients such as IL-2, GlutaMAX, or NAC, both donor cells cultured in the T+X medium maintained high viability. Due to the source of donor cells, a medium containing only X-VIVO 15 was not suitable for culture of Donor 1 cells, while a medium containing only TexMACS struggled to maintain high viability of Donor 2 cells during the later stages of culture.

TABLE 4
Proportion of CD25⁺CD69⁺ T cells (%)

| No | Day 0 | Day 1 | Day 5 | Day 7 |
|---|---|---|---|---|
| 1-1 | 0.27 | 62.2 | 55.27 | 21.63 |
| 1-2 | 0.27 | 49.55 | 29.56 | NA |
| 1-3 | 0.27 | 67.41 | 49.71 | 54.1 |
| 2-1 | 0.17 | 54.79 | 73.87 | 10.04 |
| 2-2 | 0.17 | 43.34 | 88.59 | 20.9 |
| 2-3 | 0.17 | 54.95 | 70.21 | 24.26 |
| 2-4 | 0.17 | 62.59 | 92 | 54.44 |

Note:
NA stands for cell death.

Analysis of the expression levels of activation markers in two donor T cells revealed that the proportion of CD25⁺CD69⁺ double-positive T cells in the T+X medium was higher than that in the single medium, indicating that T cells in the T+X medium exhibited a better activation state, which was conducive to T cell expansion.

TABLE 5
Proportion of early memory phenotype T_{SCM} (%)

| No | Day 0 | Day 1 | Day 3 | Day 5 | Day 7 | Day 9 | Day 11 | Day 13 |
|---|---|---|---|---|---|---|---|---|
| 1-1 | 48.78 | 59.35 | 73.85 | 81.27 | 80.45 | 81.18 | 76.07 | 61.39 |
| 1-2 | 48.78 | 88.68 | 88.35 | 79.46 | 64.28 | NA | NA | NA |
| 1-3 | 48.78 | 71.55 | 83.86 | 91.16 | 82.73 | 76.47 | 68.86 | 63.53 |
| 2-2 | 74.38 | 79.08 | 84.77 | 85.51 | 83.11 | 79.83 | 75.5 | 66.77 |
| 2-3 | 74.38 | 92.89 | 90.81 | 90.1 | 83.34 | 76.47 | 61.1 | 39.14 |
| 2-4 | 74.38 | 81.11 | 86.04 | 90.46 | 89.11 | 70.62 | 62.06 | 60.78 |

Note:
NA stands for cell death.

Further analysis of the differentiation phenotype revealed that T cells cultured in the T+X medium exhibited a higher proportion of early memory phenotype T_{SCM} and maintained a lower differentiation state, comparable to the medium containing only TexMACS.

### Example 2: Effect of Proportion of Serum-Free Medium on T Cell Culture

Human leukapheresis products were washed by centrifugation, followed by incubation with anti-CD4 magnetic beads and CD8 magnetic beads. The incubated products were sorted using a magnetic sorting system, and the sorted T cells were subsequently cryopreserved. T cells from two different donors (Donor 3 and Donor 4) were thawed, grouped after staining and counting, and washed twice by centrifugation in Medium-1. T cell pellet was resuspended in Medium-1, and the cells were assayed for density, viability, and proportion of PD-1 expression (recorded as Day 0). T cells were added to Medium-2 at a cell density of no less than 2.5×10⁶ cells/mL, and activated using magnetic beads conjugated to anti-CD3 antibody and anti-CD28 antibody. The cells were then incubated in an incubator at 37.0±1.0° C. with 5.0±1.0% CO₂ for 24±6 hours.

Activated T cells were assayed for density, viability, and proportion of PD-1 expression. Transduced T cells were supplemented with Medium-3 to the same volume per group. The cells were transferred to an incubator at 37.0±1.0° C. with 5.0±1.0% CO₂ for incubation, and T cells were assayed and supplemented with Medium-3 at the corresponding time points. Starting from Day 5, cells were passaged with a cell density of approximately 0.625×10⁶ cells/mL per passage.

The Medium-1, Medium-2, and Medium-3 were prepared according to Table 6. Specifically, the concentration of cytokines, GlutaMAX, or NAC was measured based on the volume of the medium before the addition of cytokines, GlutaMAX, or NAC.

TABLE 6
Medium formula

| | | Medium-2 | | | | | |
|---|---|---|---|---|---|---|---|
| | | Medium-3 | | | | | |
| | | Medium-1 | IL-2 | IL-7 | IL-15 | GlutaMAX | NAC |
| No. | Donor | (v:v) | (IU/mL) | (IU/mL) | (IU/mL) | (mM) | (mg/mL) |
| 3-1 | 3 | T + X (1:1) | 100 IU/mL | 1400 IU/mL | 46 IU/mL | 2 mM | 0.8 mg/mL |
| 3-2 | 3 | T + X (2:1) | | | | | |
| 3-3 | 3 | T + X (4:1) | | | | | |
| 3-4 | 3 | T + X (9:1) | | | | | |
| 4-1 | 4 | T + X (1:1) | | | | | |
| 4-2 | 4 | T + X (2:1) | | | | | |
| 4-3 | 4 | T + X (4:1) | | | | | |
| 4-4 | 4 | T + X (9:1) | | | | | |

The parameters of cumulative fold expansion, viability, and proportion of PD-1 expression during T cell culture are shown in the table below.

TABLE 7
Cumulative fold expansion

| No. | Day 0 | Day 1 | Day 3 | Day 5 | Day 7 | Day 9 | Day 11 |
|---|---|---|---|---|---|---|---|
| 3-1 | 1.00 | 0.52 | 0.99 | 3.11 | 7.88 | 34.47 | 152.04 |
| 3-2 | 1.00 | 0.55 | 1.08 | 2.97 | 8.00 | 38.57 | 177.71 |
| 3-3 | 1.00 | 0.52 | 1.09 | 3.20 | 8.72 | 36.78 | 157.48 |
| 3-4 | 1.00 | 0.55 | 1.00 | 2.78 | 7.17 | 27.85 | 114.02 |
| 4-1 | 1.00 | 0.58 | 1.44 | 3.90 | 13.66 | 54.17 | 162.83 |
| 4-2 | 1.00 | 0.54 | 1.35 | 4.07 | 16.46 | 66.74 | 220.74 |
| 4-3 | 1.00 | 0.61 | 1.57 | 4.17 | 17.98 | 70.52 | 208.42 |
| 4-4 | 1.00 | 0.61 | 1.45 | 3.73 | 14.64 | 60.51 | 154.17 |

By analyzing the cumulative fold expansion, all of the media were capable of efficiently culturing cells, and a significant improvement in expansion effect was observed when the volume ratio of T+X was 4:1 to 1:1; while the optimal expansion effect was achieved when the volume ratio of T+X was 2:1.

TABLE 8
Cell viability (%)

| No. | Day 0 | Day 1 | Day 3 | Day 5 | Day 7 | Day 9 | Day 11 |
|---|---|---|---|---|---|---|---|
| 3-1 | 77 | 62.50 | 79.57 | 72.00 | 78.83 | 86.60 | 90.70 |
| 3-2 | 77 | 65.17 | 79.60 | 69.00 | 78.67 | 87.27 | 91.43 |
| 3-3 | 77 | 64.80 | 80.77 | 71.03 | 78.20 | 86.93 | 90.50 |
| 3-4 | 77 | 66.13 | 78.77 | 69.93 | 75.03 | 82.73 | 88.73 |
| 4-1 | 75.77 | 70.50 | 81.67 | 77.20 | 87.30 | 91.33 | 91.30 |
| 4-2 | 75.77 | 72.97 | 82.77 | 79.17 | 88.53 | 92.17 | 92.50 |
| 4-3 | 75.77 | 71.53 | 83.00 | 80.10 | 89.13 | 92.47 | 91.47 |
| 4-4 | 75.77 | 73.00 | 81.47 | 77.37 | 88.97 | 91.27 | 91.17 |

By analyzing the cell viability, all of the media were capable of efficiently culturing cells while maintaining high cell viability during the culture period, with the optimal effect achieved when the volume ratio of T+X was 2:1.

TABLE 9
Proportion of PD-1 expression (%)

| No. | Day 0 | Day 1 | Day 3 | Day 5 | Day 7 | Day 9 | Day 11 |
|---|---|---|---|---|---|---|---|
| 3-1 | 14.76 | 53.54 | 87.54 | 53.18 | 9.91 | 2.04 | 1.24 |
| 3-2 | 14.76 | 52.67 | 84.52 | 48.29 | 7.93 | 1.61 | 1.02 |
| 3-3 | 14.76 | 52.76 | 85.71 | 50.71 | 9.04 | 1.7 | 1.05 |
| 3-4 | 14.76 | 51.35 | 83.75 | 48.57 | 9.37 | 1.98 | 1.03 |
| 4-1 | 12.49 | 47.42 | 86.47 | 45.98 | 6.12 | 1.11 | 0.81 |
| 4-2 | 12.49 | 45.83 | 89.74 | 50.98 | 6.43 | 1.1 | 0.72 |
| 4-3 | 12.49 | 45.35 | 89.45 | 48.53 | 6.79 | 1.62 | 0.9 |
| 4-4 | 12.49 | 44.94 | 89.15 | 51.62 | 6.93 | 1.07 | 0.73 |

A low proportion of PD-1 expression indicates a lower exhaustion phenotype in cells cultured under these conditions. All of the media in this example achieved low PD-1 expression in cells, maintaining a very low exhaustion state.

### Example 3: Effect of Cytokine Concentration on T Cell Culture

Human leukapheresis products were washed by centrifugation, followed by incubation with anti-CD4 magnetic beads and CD8 magnetic beads. The incubated products were sorted using a magnetic sorting system, and the sorted T cells were subsequently cryopreserved. T cells from two different donors (Donor 5 and Donor 6) were thawed, grouped after staining and counting, and washed twice by centrifugation in Medium-1. T cell pellet was resuspended in Medium-1, and the cells were assayed for density, viability, proportion of T_{SCM}, and proportion of PD-1 expression (recorded as Day 0). T cells were added to Medium-2 at a cell density of no less than 2.5×10⁶ cells/mL, and activated using magnetic beads conjugated to anti-CD3 antibody and anti-CD28 antibody. The cells were then incubated in an incubator at 37.0±1.0° C. with 5.0±1.0% CO₂ for 24±6 hours.

Activated T cells were assayed for density, viability, proportion of T_{SCM}, and proportion of PD-1 expression. Transduced T cells were supplemented with Medium-3 to the same volume per group. The cells were transferred to an incubator at 37.0±1.0° C. with 5.0±1.0% CO₂ for incubation, and T cells were assayed and supplemented with Medium-3 at the corresponding time points. Starting from Day 5, cells were passaged with a cell density of approximately 0.625×10⁶ cells/mL per passage.

The Medium-1, Medium-2, and Medium-3 were prepared according to Table 10. Specifically, the medium substrate of Medium-2 and Medium-3 was Medium-1 of the corresponding group, and the concentration of cytokines, GlutaMAX, or NAC was measured based on the volume of Medium-1 before the addition of cytokines, GlutaMAX, and NAC.

TABLE 10
Medium formula

| | | Medium-2 | | | | | |
|---|---|---|---|---|---|---|---|
| | | Medium-3 | | | | | |
| | | Medium-1 | IL-2 | IL-7 | IL-15 | GluMax | NAC |
| Group | Donor | (v:v) | (IU/mL) | (IU/mL) | (IU/mL) | (mM) | (mg/mL) |
| G1 | 6 | T + X | 60 | 450 | 28 | 2 | 0.8 |
| G2 | 6 | (2:1) | 20 | 700 | 10 | | |
| G3 | 6 | | 20 | 200 | 10 | | |
| G4 | 5 | | 100 | 700 | 10 | | |
| G5 | 5 | | 20 | 700 | 46 | | |
| G6 | 6 | | 20 | 200 | 46 | | |
| G7 | 5 | | 60 | 450 | 28 | | |
| G8 | 5 | | 20 | 200 | 46 | | |
| G9 | 6 | | 100 | 200 | 46 | | |
| G10 | 6 | | 60 | 450 | 28 | | |
| G11 | 6 | | 100 | 200 | 10 | | |
| G12 | 6 | | 20 | 700 | 46 | | |
| G13 | 5 | | 100 | 200 | 46 | | |
| G14 | 5 | | 20 | 200 | 10 | | |
| G15 | 5 | | 60 | 450 | 28 | | |
| G16 | 6 | | 100 | 700 | 10 | | |
| G17 | 5 | | 20 | 700 | 10 | | |
| G18 | 5 | | 100 | 200 | 10 | | |
| G19 | 5 | | 100 | 700 | 46 | | |
| G20 | 6 | | 100 | 700 | 46 | | |

The parameters of cumulative fold expansion, viability, proportion of T_{SCM}, and proportion of PD-1 expression during T cell culture are shown in the table below.

TABLE 11
Summary of T cell culture assay indicators

| | Day 11 | | | |
|---|---|---|---|---|
| | | | Proportion | |
| | Cumulative fold | Viability | of T_{SCM} | PD-1 expression |
| Group | expansion | (%) | (%) | (%) |
| G1 | 109.42 | 91.37 | 56.13 | 0.6 |
| G2 | 81.29 | 89.30 | 63.41 | 0.72 |
| G3 | 50.79 | 89.17 | 62.18 | 0.98 |
| G4 | 131.56 | 89.60 | 65.49 | 0.81 |
| G5 | 67.00 | 86.43 | 59.23 | 1.16 |
| G6 | 56.25 | 89.63 | 60.15 | 0.62 |
| G7 | 122.14 | 89.87 | 59.66 | 0.93 |
| G8 | 41.08 | 82.50 | 57.94 | 1.7 |
| G9 | 123.24 | 92.17 | 51.7 | 0.52 |
| G10 | 105.54 | 92.03 | 55.46 | 0.62 |
| G11 | 113.16 | 92.23 | 52.02 | 0.6 |
| G12 | 84.42 | 90.80 | 60.58 | 0.55 |
| G13 | 156.76 | 91.53 | 57.53 | 0.88 |
| G14 | 62.68 | 88.07 | 47.95 | 1.56 |
| G15 | 107.65 | 89.17 | 62.95 | 1 |
| G16 | 137.29 | 92.63 | 49.81 | 0.47 |
| G17 | 74.88 | 87.67 | 58.85 | 1.14 |
| G18 | 129.40 | 90.23 | 55.6 | 0.96 |
| G19 | 166.81 | 90.83 | 56.96 | 0.81 |
| G20 | 122.92 | 92.33 | 51.37 | 0.52 |

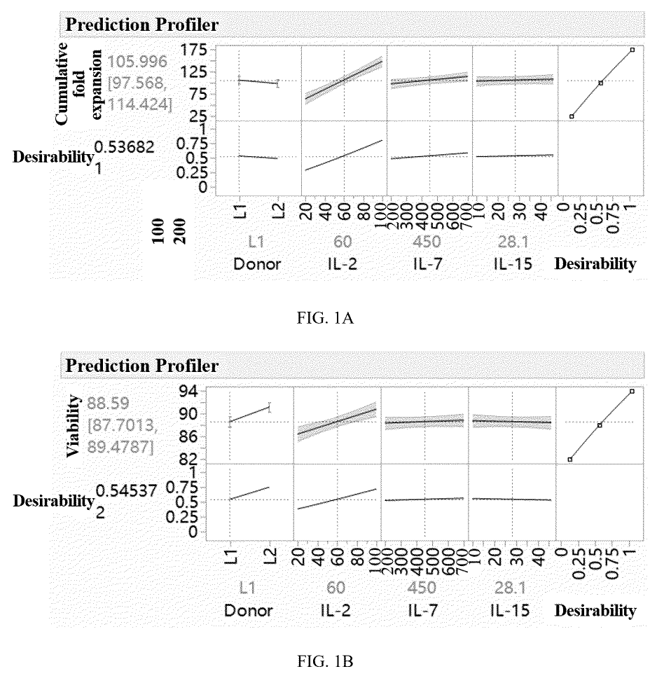

Analysis of the data is shown in FIG. 1A to FIG. 1D. An increase in the concentration of IL-2 is beneficial for achieving efficient cell expansion and maintaining high cell viability, and to some extent can increase the proportion of T_{SCM} and reduce the proportion of PD-1 expression. When the concentration of IL-2 reached 60 IU/mL or higher, good cell culture effect was achieved. The concentrations of IL-7 and IL-15 showed no significant effect on the cumulative fold expansion or cell viability. An increase in the concentration of IL-7 to some extent can increase the proportion of T_{SCM} and reduce the proportion of PD-1 expression; while IL-15 showed no significant effect on these two indicators.

### Example 4: Effect of Combination of Various Media and TexMACS on T Cell Culture

Human leukapheresis products were washed by centrifugation, followed by incubation with anti-CD4 magnetic beads and CD8 magnetic beads. The incubated products were sorted using a magnetic sorting system, and the sorted T cells were subsequently cryopreserved. T cells from two different donors (Donor 7 and Donor 8) were thawed, grouped after staining and counting, and washed twice by centrifugation in DPBS (Corning, 21-031-CV). T cell pellet was resuspended in DPBS, and the cells were assayed for viability. T cells were cultured in a G-Rex 24 culture plate, added with Medium-2 to a cell density of no less than 2.5×10⁶ cells/mL, and activated using magnetic beads conjugated to anti-CD3 antibody and anti-CD28 antibody. The cells were then incubated in an incubator at 37.0±1.0° C. with 5.0±1.0% CO₂ for 24±6 hours.

Activated T cells were supplemented with Medium-3 to the same volume per group. The cells were transferred to an incubator at 37.0±1.0° C. with 5.0±1.0% CO₂ for incubation, and T cells were assayed and supplemented with Medium-3 at the corresponding time points. Starting from Day 5, cells were passaged with a cell density of approximately 0.625×10⁶ cells/mL per passage.

The Medium-1 was prepared according to Table 12. Medium-2 was Medium-1 supplemented with 100 IU/mL IL-2, 700 IU/mL IL-7, 46 IU/mL IL-15, 2 mM GlutaMAX, and 0.8 mg/mL NAC; Medium-3 was Medium-1 supplemented with 100 IU/mL IL-2, 700 IU/mL IL-7, 46 IU/mL IL-15, and 2 mM GlutaMAX. The concentration of cytokines, GlutaMAX, or NAC was measured based on the volume of Medium-1 before the addition of cytokines, GlutaMAX, and NAC. The parameters of cumulative fold expansion, viability, proportion of early memory phenotype T_{SCM}, and proportion of PD-1 expression on Day 13 of T cell culture are shown in Table 12.

TABLE 12
Medium formula and expansion efficiency

| | | | Cumulative | | Proportion | PD-1 |
|---|---|---|---|---|---|---|
| No | Donor | Medium-1 (v:v) | fold expansion | Viability/% | of T_{SCM}/% | expression/% |
| 7-1 | 7 | TexMACS:X-VIVO 10 = 2:1 | 91.69 | 85.13 | 75.36 | 1.15 |
| 7-2 | 7 | TexMACS:X-VIVO 15 = 2:1 | 73.72 | 81.83 | 65.15 | 2.96 |
| 7-3 | 7 | TexMACS:X-VIVO 20 = 2:1 | 68.24 | 80.83 | 77.19 | 1.72 |
| 7-4 | 7 | TexMACS:OpTmizer = 2:1 | NA | NA | NA | NA |
| 7-5 | 7 | TexMACS:AIM-V = 2:1 | NA | NA | NA | NA |
| 7-6 | 7 | TexMACS:RPMI 1640 = 2:1 | 9.22 | 64.53 | 80.92 | 1.62 |
| 7-7 | 7 | TexMACS:DMEM = 2:1 | 9.94 | 38.33 | 70.66 | 3.36 |
| 7-8 | 7 | TexMACS:MEM = 2:1 | NA | NA | NA | NA |
| 7-9 | 7 | TexMACS | 10.20 | 42.60 | 72.91 | 2.79 |
| 7-10 | 7 | X-VIVO 10 | 12.90 | 76.67 | 92.36 | 1.78 |
| 7-11 | 7 | X-VIVO 15 | 9.19 | 70.60 | 69.32 | 16.45 |
| 7-12 | 7 | X-VIVO 20 | 41.11 | 85.50 | 73.63 | 6.98 |
| 7-13 | 7 | OpTmizer | 11.48 | 48.23 | 87.72 | 3.86 |
| 7-14 | 7 | AIM-V | NA | NA | NA | NA |
| 7-15 | 7 | RPMI 1640 | NA | NA | NA | NA |
| 7-16 | 7 | DMEM | NA | NA | NA | NA |
| 7-17 | 7 | MEM | NA | NA | NA | NA |
| 8-1 | 8 | TexMACS:X-VIVO 10 = 2:1 | 83.10 | 83.33 | 74.57 | 2.25 |
| 8-2 | 8 | TexMACS:X-VIVO 15 = 2:1 | 73.97 | 82.67 | 63.66 | 4.41 |
| 8-3 | 8 | TexMACS:X-VIVO 20 = 2:1 | 90.25 | 83.10 | 73.35 | 2.73 |
| 8-4 | 8 | TexMACS:OpTmizer = 2:1 | NA | NA | NA | NA |
| 8-5 | 8 | TexMACS:AIM-V = 2:1 | NA | NA | NA | NA |
| 8-6 | 8 | TexMACS:RPMI 1640 = 2:1 | 5.69 | 42.73 | 74.06 | 2.27 |
| 8-7 | 8 | TexMACS:DMEM = 2:1 | 10.79 | 42.70 | 59.53 | 6.4 |
| 8-8 | 8 | TexMACS:MEM = 2:1 | NA | NA | NA | NA |
| 8-9 | 8 | TexMACS | 16.88 | 51.17 | 64.1 | 3.42 |
| 8-10 | 8 | X-VIVO 10 | 18.46 | 78.60 | 90.16 | 2.65 |
| 8-11 | 8 | X-VIVO 15 | 9.06 | 79.73 | 63.31 | 9.59 |
| 8-12 | 8 | X-VIVO 20 | 40.96 | 81.97 | 73.64 | 6.42 |
| 8-13 | 8 | OpTmizer | 7.63 | 38.60 | 89.39 | 1.74 |
| 8-14 | 8 | AIM-V | NA | NA | NA | NA |
| 8-15 | 8 | RPMI 1640 | NA | NA | NA | NA |
| 8-16 | 8 | DMEM | NA | NA | NA | NA |
| 8-17 | 8 | MEM | NA | NA | NA | NA |

Note:
NA indicates that cells died during culture and could not be cultured until Day 13.

Fold expansion is a key indicator of medium efficiency. The combined medium obtained by mixing TexMACS with X-VIVO 10, X-VIVO 15, or X-VIVO 20 significantly outperformed the combined medium obtained by mixing TexMACS with OpTmizer, AIM-V, RPMI 1640, DMEM, or MEM, as well as the single medium in terms of fold expansion. Surprisingly, TexMACS exhibited no synergistic effect at all with other media except for the combination of TexMACS with X-VIVO 10, X-VIVO 15, or X-VIVO 20. Meanwhile, the combined medium obtained by mixing TexMACS with X-VIVO 10, X-VIVO 15, or X-VIVO 20 maintained high cell viability, with the proportion of early memory phenotype T_{SCM} and the proportion of exhaustion marker PD-1 expression similar to those in other groups.

The effect of other components on the medium composition was also evaluated in this study. The T cell collection and culture process for Donor 8 was the same as above, with Medium-1 prepared according to Table 13, wherein T+X (2:1) indicates a volume ratio of 2:1 for the mixture of TexMACS and X-VIVO 15. Medium-2 was Medium-1 supplemented with 100 IU/mL IL-2, 700 IU/mL IL-7, 46 IU/mL IL-15, 2 mM GlutaMAX, and 0.8 mg/mL NAC; Medium-3 was Medium-1 supplemented with 100 IU/mL IL-2, 700 IU/mL IL-7, 46 IU/mL IL-15, and 2 mM GlutaMAX. The concentration of cytokines, GlutaMAX, or NAC was measured based on the volume of Medium-1 before the addition of cytokines, GlutaMAX, and NAC. The parameters of cumulative fold expansion, viability, proportion of early memory phenotype T_{SCM}, and proportion of PD-1 expression on Day 13 of T cell culture are shown in Table 13.

TABLE 13
Medium formula and expansion efficiency

| | | | Cumulative | | Proportion | PD-1 |
|---|---|---|---|---|---|---|
| No | Donor | Medium-1 (v:v) | fold expansion | Viability/% | of T_{SCM/}% | expression/% |
| 8-2 | 8 | T + X (2:1) | 73.97 | 82.67 | 63.66 | 4.41 |
| 8-9 | 8 | TexMACS | 16.88 | 51.17 | 64.1 | 3.42 |
| 8-11 | 8 | X-VIVO 15 | 9.06 | 79.73 | 63.31 | 9.59 |
| 8-18 | 8 | T + X (2:1) 80% + | 41.05 | 83.57 | 62.67 | 2.04 |
| | | DPBS 20% | | | | |
| 8-19 | 8 | T + X (2:1) 80% + | 89.50 | 87.80 | 65.2 | 5.2 |
| | | AIM-V 20% | | | | |
| 8-20 | 8 | T + X (2:1) 65% + | 38.94 | 76.17 | 66.95 | 2.25 |
| | | DPBS 35% | | | | |
| 8-21 | 8 | T + X (2:1) 65% + | 28.75 | 85.03 | 57.98 | 9.32 |
| | | AIM-V 35% | | | | |

Note:
NA indicates that cells died during culture and could not be cultured until Day 13.

The combined medium of T+X (2:1) can be moderately diluted, and the diluted medium still significantly outperforms the single medium. The combined medium of TexMACS, X-VIVO 15, and AIM-V can achieve a superior expansion effect. When the combined medium of T+X (2:1) was mixed with AIM-V medium at a volume ratio of 4:1, the fold expansion and cell viability of T cells were higher than those of the T+X (2:1) medium, while the proportion of early memory phenotype T_{SCM} and the proportion of exhaustion marker PD-1 expression were comparable to those of the T+X (2:1) medium.

### Example 5: Effect of Proportion of Three-Phase Medium on T Cell Culture

Human leukapheresis products were washed by centrifugation, followed by incubation with anti-CD4 magnetic beads and CD8 magnetic beads. The incubated products were sorted using a magnetic sorting system, and the sorted T cells were subsequently cryopreserved. T cells from two different donors (Donor 9 and Donor 10) were thawed, grouped after staining and counting, and washed twice by centrifugation in DPBS. T cell pellet was resuspended in DPBS, and the cells were assayed for viability. T cells were cultured in a G-Rex 24 culture plate, added with Medium-2 to a cell density of no less than 2.5×10⁶ cells/mL, and activated using magnetic beads conjugated to anti-CD3 antibody and anti-CD28 antibody. The cells were then incubated in an incubator at 37.0±1.0° C. with 5.0±1.0% CO₂ for 24±6 hours.

Activated T cells were supplemented with Medium-3 to the same volume per group. The cells were transferred to an incubator at 37.0±1.0° C. with 5.0±1.0% CO₂ for incubation, and T cells were assayed and supplemented with Medium-3 at the corresponding time points. Starting from Day 5, cells were passaged with a cell density of approximately 0.625×10⁶ cells/mL per passage.

The Medium-1 was prepared according to Table 14. Medium-2 was Medium-1 supplemented with 100 IU/mL IL-2, 700 IU/mL IL-7, 46 IU/mL IL-15, 2 mM GlutaMAX, and 0.8 mg/mL NAC; Medium-3 was Medium-1 supplemented with 100 IU/mL IL-2, 700 IU/mL IL-7, 46 IU/mL IL-15, and 2 mM GlutaMAX. The concentration of cytokines, GlutaMAX, or NAC was measured based on the volume of Medium-1 before the addition of cytokines, GlutaMAX, and NAC. The parameters of cumulative fold expansion, viability, proportion of early memory phenotype T_{SCM}, and proportion of PD-1 expression on Day 13 of T cell culture are shown in Table 14.

TABLE 14
Medium formula and expansion efficiency

| | | | | | | | | PD-1 |
|---|---|---|---|---|---|---|---|---|
| | | Medium-1 (v:v:v) | | | Cumulative | Viability/ | Proportion | expression/ |
| No | Donor | TexMACS | X-VIVO 15 | AIM-V | fold expansion | % | of T_{SCM}/% | % |
| 9-1 | 9 | 0.67 | 0.33 | 0 | 615.75 | 91.23 | 29.73 | 8.55 |
| 9-2 | 9 | 0.7 | 0.2 | 0.1 | 646.13 | 91.57 | 29.09 | 7.35 |
| 9-3 | 9 | 0.5 | 0.4 | 0.1 | 688.64 | 91.77 | 25.8 | 9.38 |
| 9-4 | 9 | 0.5 | 0.25 | 0.25 | 585.16 | 91.23 | 27.59 | 9.12 |
| 9-5 | 9 | 0.3 | 0.4 | 0.3 | 249.62 | 89.90 | 26.31 | 9.94 |
| 10-1 | 10 | 0.67 | 0.33 | 0 | 321.50 | 91.27 | 30.42 | 5.56 |
| 10-2 | 10 | 0.7 | 0.1 | 0.2 | 273.34 | 90.70 | 30.03 | 4.67 |
| 10-3 | 10 | 0.5 | 0.4 | 0.1 | 423.49 | 91.53 | 26.09 | 6.12 |
| 10-4 | 10 | 0.5 | 0.25 | 0.25 | 433.37 | 91.70 | 26.25 | 5.21 |
| 10-5 | 10 | 0.3 | 0.3 | 0.4 | 367.77 | 90.87 | 25.63 | 6.01 |

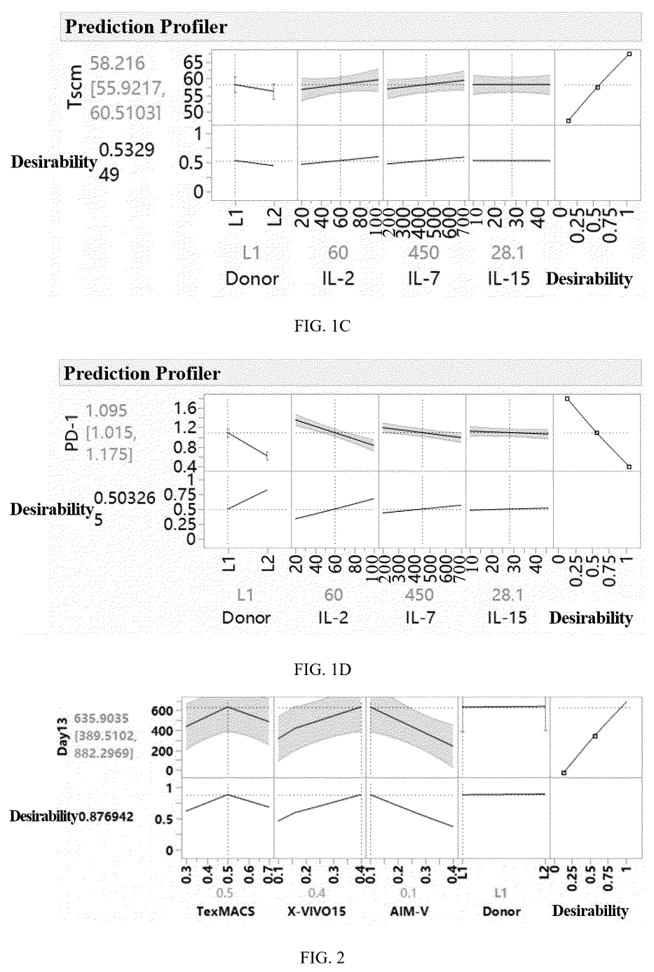

Analysis of the cumulative fold expansion data is shown in FIG. 2. Data fitting analysis revealed an optimal ratio of 5:4:1 for the combination of TexMACS, X-VIVO 15, and AIM-V. By comparing experimental data from the same donors (Group 9-1 vs. Group 9-3 and Group 10-1 vs. Group 10-3), at the optimal ratio, the fold expansion of T cells was significantly higher than that of the combined medium of T+X (2:1); the cell viability, the proportion of early memory cells, and the proportion of exhaustion marker PD-1 expression were comparable to those of the combined medium of T+X (2:1).

1. A composition, wherein:
(1) the composition is composition 1, comprising a first serum-free medium and a second serum-free medium at a volume ratio of 1:(0.1 to 10); the composition has a higher fold for expansion of immune cells than either the first serum-free medium or the second serum-free medium; or,
(2) the composition is composition 2, comprising a medium substrate, wherein the medium substrate comprises a first serum-free medium and a second serum-free medium; the sum of the volumes of the first serum-free medium and the second serum-free medium accounts for 65% or more, or 80% or more of the volume of the medium substrate, or the medium substrate consists of a first serum-free medium and a second serum-free medium; the volume ratio of the first serum-free medium to the second serum-free medium is 1:(0.1 to 10); the first serum-free medium is TexMACS; the second serum-free medium consists of one or more media selected from the group consisting of X-VIVO 10, X-VIVO 15, and X-VIVO 20.

2. (canceled)

3. The composition according to claim 1, wherein the volume ratio of the first serum-free medium to the second serum-free medium is 1:(0.1 to 2); preferably, the volume ratio of the first serum-free medium to the second serum-free medium is 1:(0.25 to 1).

4. The composition according to claim 1, wherein in composition 2, the medium substrate comprises a third serum-free medium AIM-V; the ratio of the sum of the volumes of the first and second serum-free media to the volume of the third serum-free medium is not less than 2:1.

5. The composition according to claim 1, wherein in composition 2, the composition comprises a supplement added to the medium substrate; the supplement comprises one or more components selected from the group consisting of inorganic salts, sugars, vitamins, albumin, lipids, amino acids, cytokines, and antioxidants; preferably, the supplement comprises one or more components selected from the group consisting of amino acids, cytokines, and antioxidants; more preferably, the supplement comprises amino acids, cytokines, and antioxidants.

6. The composition according to claim 5, wherein the cytokine comprises one or more components selected from the group consisting of IL-1, IL-2, IL-3, IL-4, IL-6, IL-7, IL-9, IL-10, IL-12, IL-13, IL-15, IL-17, IL-18, IL-21, IL-22, interferon, and tumor necrosis factor; preferably, the cytokine comprises one or more components selected from the group consisting of IL-2, IL-7, and IL-15.

7. The composition according to claim 6, wherein the concentration of the IL-2 supplementarily added in the composition is 10 to 1000 IU/mL.

8. The composition according to claim 5, wherein the amino acid is L-alanyl-L-glutamine; preferably, the concentration of the L-alanyl-L-glutamine supplementarily added in the composition is 1 mM to 5 mM; more preferably, the concentration of the L-alanyl-L-glutamine supplementarily added in the composition is about 2 mM.

9. The composition according to claim 5, wherein the antioxidant is N-acetyl-L-cysteine; preferably, the concentration of the N-acetyl-L-cysteine supplementarily added in the composition is 0.5 mg/mL to 2 mg/mL; more preferably, the concentration of the N-acetyl-L-cysteine supplementarily added in the composition is about 0.8 mg/mL.

10. The composition according to claim 1, wherein the composition 2 comprises a medium substrate, wherein the medium substrate comprises the first serum-free medium, the second serum-free medium, and optionally a third serum-free medium AIM-V; the sum of the volumes of the first serum-free medium and the second serum-free medium accounts for 80% or more of the volume of the medium substrate; the volume ratio of the first serum-free medium to the second serum-free medium is 1:(0.25 to 1);
preferably, the medium substrate consists of the first serum-free medium and the second serum-free medium, wherein the volume ratio of the first serum-free medium to the second serum-free medium is 1:(0.25 to 1); or the medium substrate consists of the first serum-free medium, the second serum-free medium, and the third serum-free medium, wherein the volume ratio of the first serum-free medium to the second serum-free medium is 1:(0.25 to 1), and the ratio of the sum of the volumes of the first and second serum-free media to the volume of the third serum-free medium is not less than 10:1.

11. The composition according to claim 10, comprising a supplement added to the medium substrate, wherein the supplement comprises IL-2, IL-7, IL-15, L-alanyl-L-glutamine, and optionally N-acetyl-L-cysteine.

12. A method for preparing a medium, comprising mixing the medium substrate in the composition according to claim 5 with a supplement.

13. A method for culturing cells, comprising incubating the cell in the composition according to claim 1; preferably, the cell is a myeloid or lymphoid cell; more preferably, the cell is a T cell.

14. A cell produced by the method for culturing cells according to claim 13.

15. The composition according to claim 3, wherein the volume ratio of the first serum-free medium to the second serum-free medium is about 1:0.5.

16. The composition according to claim 4, wherein the ratio of the sum of the volumes of the first and second serum-free media to the volume of the third serum-free medium is not less than 4:1.

17. The composition according to claim 4, wherein the ratio of the sum of the volumes of the first and second serum-free media to the volume of the third serum-free medium is not less than 10:1.

18. The composition according to claim 7, wherein the concentration of the IL-2 supplementarily added in the composition is 60 to 200 IU/mL; preferably, the concentration of the IL-2 supplementarily added in the composition is about 100 IU/mL.

19. The composition according to claim 10, wherein the medium substrate consists of the first serum-free medium and the second serum-free medium, wherein the volume ratio of the first serum-free medium to the second serum-free medium is about 1:0.5; or the medium substrate consists of the first serum-free medium, the second serum-free medium, and the third serum-free medium, wherein the volume ratio of the first serum-free medium, the second serum-free medium, and the third serum-free medium is about 5:4:1.

20. The composition according to claim 11, wherein the concentration of the IL-2 supplementarily added in the composition is 50 to 200 IU/mL, the concentration of the IL-7 is 500 to 1000 IU/mL, the concentration of the IL-15 is 25 to 100 IU/mL, the concentration of the L-alanyl-L-glutamine is 1 mM to 5 mM, and the concentration of the N-acetyl-L-cysteine is 0.5 mg/mL to 2 mg/mL.

21. The composition according to claim 11, wherein the concentration of the IL-2 supplementarily added in the composition is about 100 IU/mL, the concentration of the IL-7 is about 700 IU/mL, the concentration of the IL-15 is about 46 IU/mL, the concentration of the L-alanyl-L-glutamine is about 2 mM, and the concentration of the N-acetyl-L-cysteine is about 0.8 mg/mL.
